SEQUENCE LISTING (22 sequences)

SEQ ID NO: 1 - protein, 248 aa, Hepatitis C virus
GTASATMNPLLILTFVAAALAETHQNIQLINTNGSWHINSTALNCNESLNTGWLAGLFYQHKFNSSGCPERLASCGSSGCWHYPPRPCGIVPAKSVCGPVYCFTPSPVVVGTTDRSGAPTYSWGANDTDVFVLNNTRPPLGNWFGCTWMNSTGFTKVCGAPPCGSSGCPTDCFRKHPEATYSRCGSGPWITPRCMVDYPYRLWHYPCTINYTIFKVRMYVGGVEHRLEAACNWTRGERCDLEDRDRSE

SEQ ID NO: 2 - DNA, 744 bp, Hepatitis C virus
ggtaccgctagcgccaccatgaaccccctgctgatcctgaccttcgtggctgcagccctggccgagacccaccagaacatccagctgatcaacaccaacggcagctggcacatcaacagcaccgccctgaactgcaacgagagcctgaacaccggctggctggccggcctgttctaccagcacaagttcaacagcagcggctgccccgagaggctcgcctcctgcggcagcagcggctgctggcactacccccccagaccctgcggcatcgtgcccgccaagagcgtgtgcggccctgtgtactgcttcacccccagccccgtggtggtgggcaccaccgacagaagcggagcccccacctacagctggggcgccaacgacaccgacgtgttcgtgctgaacaacaccagaccccccctgggcaactggttcggctgcacctggatgaacagcaccggcttcaccaaagtgtgtggcgcccctccctgcggcagcagcggctgccccaccgactgctttaggaaacaccccgaggccacctactccagatgcggcagcggcccctggatcaccccccggtgcatggtggactacccctaccggctgtggcactatccctgcaccatcaactacaccatcttcaaagtgcggatgtacgtgggaggcgtcgagcataggctggaagcagcttgcaattggacaaggggcgagcggtgcgacctggaagatcgggaccgcagcgag

SEQ ID NO: 3 - DNA, 40551 bp, Artificial
  ChAdOx1-TPA-E2D123 sequence
ccaccgcctcggacctggccgacctcatcttccccgagcgcctcaggctgacgctgcgcaacggcctgcccgactttatgagccaaagcatgttgcaaaaactttcggtaaaaaatgtaattactccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatataagccctaacctacactgacgtaaaagggcccaaaagtctaaaaaaataacccgcccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacrayacgagtgggaycgtggtcccagactaataatcagaccgacgatacgagtgggaccgtggtcccagactaataatcagaccgacgatacgagtgggaccgtggtyccagwctratwatcagaccgacgatacragtggracmgtggkcccagasakaatawtcagrccgagwtaygcwktckggcctgtaacaaaggacattaagtaaagacagatamrmgtgrgactaaaacgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaaccaattcagtcgagcctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggttaagcttggtacccccgccgccaccatggacgctatgaagcgaggactgtgctgcgtgctgctgctgtgtggcgctgtgtttgtgtcccctagccaagagatccacgccagattcagaagagagacacaccagaacatccagctgatcaacaccaacggcagctggcacatcaacagcacagccctgaactgcaacgagagcctgaataccggatggctggccggcctgttctaccagcacaagttcaatagcagcggctgccccgagagactggccagctgtggatcttctggctgctggcactaccctccaagaccttgtggaatcgtgcccgccaagtctgtgtgtggccccgtgtactgcttcaccccatctccagtggtcgtgggcaccaccgatagatctggcgccccaacatatagctggggagccaacgacaccgacgtgttcgtgctgaacaataccagacctcctctcggcaattggttcggctgcacctggatgaacagcaccggcttcacaaaagtgtgcggagcccctccatgtggcagctctggatgtcctaccgactgcttcagaaagcaccccgaggccacctacagcagatgtggatctggcccttggatcacccctcggtgcatggtggactacccctacagactgtggcactatccctgcaccatcaactacaccatcttcaaagtgcggatgtacgtcggcggcgtggaacacagactggaagccgcctgtaattggaccagaggcgagagatgcgacctcgaggacagagacagatccgaatgagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaataattgagtctgacaaaaggacttgatgtg

SEQ ID NO: 4 - DNA, 9887 bp, Artificial
  ChAdOx1 sequence 5' to the TPA-E2D123 immunogen cassette
ccaccgcctcggacctggccgacctcatcttccccgagcgcctcaggctgacgctgcgcaacggcctgcccgactttatgagccaaagcatgttgcaaaaactttcggtaaaaaatgtaattactccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatataagccctaacctacactgacgtaaaagggcccaaaagtctaaaaaaataacccgcccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactatagggtcaccgtcgacagcgacacacttgcatcggatgcagcccggttaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacrayacgagtgggaycgtggtcccagactaataatcagaccgacgatacgagtgggaccgtggtcccagactaataatcagaccgacgatacgagtgggaccgtggtyccagwctratwatcagaccgacgatacragtggracmgtggkcccagasakaatawtcagrccgagwtaygcwktckggcctgtaacaaaggacattaagtaaagacagatamrmgtgrgactaaaacgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccctgtcgctcaactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgacggcgcgccaaagcttgcatgcctgcagccgcgtaacctggcaaaatcggttacggttgagtaataaatggatgccctgcgtaagcggggcacatttcattacctctttctccgcacccgacatagataataacttcgtatagtatacattatacgaagttatctagtagacttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagcaggctccaccatgggaaccaattcagtcgag

SEQ ID NO: 5 - DNA, 673 bp, Artificial
  TPA-E2D123 immunogen cassette sequence
cctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggt

SEQ ID NO: 6 - DNA, 13 bp, Artificial
  Extra sequence
taagcttggtacc

SEQ ID NO: 7 - DNA, 12 bp, Artificial
  Kozak sequence
cccgccgccacc

SEQ ID NO: 8 - DNA, 96 bp, Artificial
  TPA sequence
atggacgctatgaagcgaggactgtgctgcgtgctgctgctgtgtggcgctgtgtttgtgtcccctagccaagagatccacgccagattcagaaga

SEQ ID NO: 9 - protein, 32 aa, Artificial
  TPA amino acid sequence
MDAMKRGLCCVLLLCGAVFVSPSQEIHARFRR

SEQ ID NO: 10 - DNA, 684 bp, Artificial
  E2D123 sequence
gagacacaccagaacatccagctgatcaacaccaacggcagctggcacatcaacagcacagccctgaactgcaacgagagcctgaataccggatggctggccggcctgttctaccagcacaagttcaatagcagcggctgccccgagagactggccagctgtggatcttctggctgctggcactaccctccaagaccttgtggaatcgtgcccgccaagtctgtgtgtggccccgtgtactgcttcaccccatctccagtggtcgtgggcaccaccgatagatctggcgccccaacatatagctggggagccaacgacaccgacgtgttcgtgctgaacaataccagacctcctctcggcaattggttcggctgcacctggatgaacagcaccggcttcacaaaagtgtgcggagcccctccatgtggcagctctggatgtcctaccgactgcttcagaaagcaccccgaggccacctacagcagatgtggatctggcccttggatcacccctcggtgcatggtggactacccctacagactgtggcactatccctgcaccatcaactacaccatcttcaaagtgcggatgtacgtcggcggcgtggaacacagactggaagccgcctgtaattggaccagaggcgagagatgcgacctcgaggacagagacagatccgaatga

SEQ ID NO: 11 - protein, 227 aa, Artificial
  E2D123 amino acid sequence
ETHQNIQLINTNGSWHINSTALNCNESLNTGWLAGLFYQHKFNSSGCPERLASCGSSGCWHYPPRPCGIVPAKSVCGPVYCFTPSPVVVGTTDRSGAPTYSWGANDTDVFVLNNTRPPLGNWFGCTWMNSTGFTKVCGAPPCGSSGCPTDCFRKHPEATYSRCGSGPWITPRCMVDYPYRLWHYPCTINYTIFKVRMYVGGVEHRLEAACNWTRGERCDLEDRDRSE

SEQ ID NO: 12 - DNA, 78 bp, Artificial
  Extra sequence
gcggccgctcgagcatgcatctagagggccctattctatagtgtcacctaaatgctagagctcgctgatcagcctcga

SEQ ID NO: 13 - DNA, 225 bp, Artificial
  BGH polyA sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 14 - DNA, 28883 bp, Artificial
  ChAdOx1 sequence 5' to the TPA-E2D123 immunogen cassette
cttctgaggcggaaagaaccagctggggctcgaggggggatcgatccgtcgagatatctagacccagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttctcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatggtttaaacgcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaataattgagtctgacaaaaggacttgatgtg

SEQ ID NO: 15 - DNA, 3293 bp, Artificial
  MVA-TPA-E2D123 sequence
gtaatctattcgatataccgttgctaacagtatactggcccaataactgtggatggaaaatctataataatacattaatatcatccgatggtgctagggttatttggatggatgcgtataaattttcttgcggtttatctttacaagactattgttatcattggggtagcaaaccagagagccgaccattcgatttaataaaaaaatcagatgctaaacgcaattctaaatcgttggtcaaagaatctatggcatccttgaaatccttgtacgaggcattcgagacacaatcaggagcgttagaagttttaatgagtccatgtaggatgttttcgttttctagaatagaagacatgttcttaactagtgtcattaatagagtatccgagaatactggaatggggatgtattatcctaccaacgatataccttctctatttatcgaatcatctatctgtctagattatattatagtaaataatcaggaatccaacaaatatcgtatcaaatctgttctcgatatcatttcttcaaaacaataccctgcaggacgtcccaactacgttaaaaatggtacaaaaggaaagttatatatcgcgttgtgtaaagttaccgtacctactaacgaccatattccagtagtttatcacgatgatgacaatactaccacctttattacagtattgacgtccgtcgatattgaaactgctatcagagcaggatattcgatagtcgaattaggggctttacaatgggataataatattccagaacttaaaaacggtttactggatagtatcaagatgatttatgacttgaacgcagttacaacaaataatttattggaacagctcatagaaaatattaactttaacaactctagtataatttcgttgttttatacatttgccattagttattgccgagcattcatttactcaattatggaaaccatagatccggtgtatatatctcagttcagttataaagaattatacgttagtagctcttataaagatattaatgaatccatgagtcagatggtaaaattataaaaagtgaaaaacaatattatttttatcgttggttgttacacttcgacattaaaaattgaaaataaatacaaaggttcttgagggttgtgttaaattgaaagcgagaaataatcataaatatgattcaggtgacggatccatggacgccatgaagcggggactgtgttgtgtgttgctgctgtgcggagccgtgttcgtgtctcccagccaagagatccacgccaggttcagaagagagacacatcagaacatccagctgatcaacaccaacggctcttggcacattaacagcaccgctctgaattgcaatgagtccctgaataccggatggctggccggactgttttaccagcacaagttcaacagcagcggctgccctgagagactggccagctgtggatctagcggatgctggcactatccacctagaccttgcggaatcgtgcccgccaaaagcgtgtgtggacccgtctattgctttacccctagtcctgtggttgtcggcacaaccgacagaagcggcgctccaacatattcctggggcgccaacgacaccgatgtgttcgtgctgaacaacacccggcctccactcggcaattggttcggctgcacctggatgaactccacaggcttcaccaaagtgtgcggcgcacctccttgtggctcctctggatgtcctaccgactgttttcggaagcaccccgaagccacatactccagatgcggatctggcccctggatcaccccaagatgtatggtggattatccttatcggctgtggcattatccgtgcacgatcaactacaccatcttcaaagtgcggatgtacgtcggcggcgtggaacacagactggaagccgcctgtaattggaccagaggcgagagatgtgatctcgaggaccgggacagatccgagtgattaaccgagtttctgcattattgtaattcgtatgctggcaccatcaaagaatcacttctaaaagatatcaatatcacacatacaaatattactaccctattgaatgagacagccaaggttatcaagttagtaaaatctctggtagataaagaagatactgatattgtgaataatttcattaccaaagaaattaaaaacagagacaaaatagttaatagtttgtctctatcaaacctggactttcgtttgtaaattggggctttttgtacaataaatgggtgttgccaatgattcatcccctgaatatcaatggatgtctccccatagattatcagatactgttatattaggagactgtttgtattttaacaatataatgtcccaattagatttacaccaaaattgggctccatcagttagattgttaaattattttaagaattttaataaggaaacactactaaagatagaagagaatgattacattaattcatcctttttccaacaaaaggataaacgattttatcctataaacgacgatttttatcacatatctacaggaggatatggtatagtctttaagatagataactatgtagtaaaatttgtattcgaggccacaaaattatatagtcccatggaaactacggcggagttcacagtacccaaatttctatacaacaatctaaagggagatgaaaaaaaattaatcgtgtgtgcgtgggccatgggattaaactataaattaacatttttacatactctgtataaacgtgttcttcatatgttgctattattgatacaaactatggatggtcaggaactatcattgagatattcttctaaagtttttttaaaggcgtttaacgagagaaaggacagtatcaaattcgtgaaattactatcccacttttatccggcagttattaacagtaatattaatgttataaactattttaaccgcatgtttcactttttcgaacatgaaaagagaactaactacgaatacgaaagaggaaatattataatttttcccctagcactgtattcggcagataaagtagataccgagctagctatcaaattaggatttaaatctttggtacaatacataaagtttatctttttacagatggctctgttatacattaaaatttacgaactaccatgctgcgacaactttttacacgcagatcttaaacccgataatatcttactttttgattccaatgaaccaataataattcatctaaaggataaaaagtttgtttttaatgaacgtattaaatcggcattaaacgactttgacttttcccaag

SEQ ID NO: 16 - DNA, 1097 bp, Artificial
  F11-L-Flank sequence
gtaatctattcgatataccgttgctaacagtatactggcccaataactgtggatggaaaatctataataatacattaatatcatccgatggtgctagggttatttggatggatgcgtataaattttcttgcggtttatctttacaagactattgttatcattggggtagcaaaccagagagccgaccattcgatttaataaaaaaatcagatgctaaacgcaattctaaatcgttggtcaaagaatctatggcatccttgaaatccttgtacgaggcattcgagacacaatcaggagcgttagaagttttaatgagtccatgtaggatgttttcgttttctagaatagaagacatgttcttaactagtgtcattaatagagtatccgagaatactggaatggggatgtattatcctaccaacgatataccttctctatttatcgaatcatctatctgtctagattatattatagtaaataatcaggaatccaacaaatatcgtatcaaatctgttctcgatatcatttcttcaaaacaataccctgcaggacgtcccaactacgttaaaaatggtacaaaaggaaagttatatatcgcgttgtgtaaagttaccgtacctactaacgaccatattccagtagtttatcacgatgatgacaatactaccacctttattacagtattgacgtccgtcgatattgaaactgctatcagagcaggatattcgatagtcgaattaggggctttacaatgggataataatattccagaacttaaaaacggtttactggatagtatcaagatgatttatgacttgaacgcagttacaacaaataatttattggaacagctcatagaaaatattaactttaacaactctagtataatttcgttgttttatacatttgccattagttattgccgagcattcatttactcaattatggaaaccatagatccggtgtatatatctcagttcagttataaagaattatacgttagtagctcttataaagatattaatgaatccatgagtcagatggtaaaattataaaaagtgaaaaacaatattatttttatcgttggttgttacact

SEQ ID NO: 17 - DNA, 97 bp, Artificial
  mH5 promoter sequence
tcgacattaaaaattgaaaataaatacaaaggttcttgagggttgtgttaaattgaaagcgagaaataatcataaatatgattcaggtgacggatcc

SEQ ID NO: 18 - DNA, 96 bp, Artificial
  TPA sequence
atggacgccatgaagcggggactgtgttgtgtgttgctgctgtgcggagccgtgttcgtgtctcccagccaagagatccacgccaggttcagaaga

SEQ ID NO: 19 - protein, 32 aa, Artificial; identical to SEQ ID NO: 9
  TPA amino acid sequence

SEQ ID NO: 20 - DNA, 684 bp, Artificial
  E2D123 sequence
gagacacatcagaacatccagctgatcaacaccaacggctcttggcacattaacagcaccgctctgaattgcaatgagtccctgaataccggatggctggccggactgttttaccagcacaagttcaacagcagcggctgccctgagagactggccagctgtggatctagcggatgctggcactatccacctagaccttgcggaatcgtgcccgccaaaagcgtgtgtggacccgtctattgctttacccctagtcctgtggttgtcggcacaaccgacagaagcggcgctccaacatattcctggggcgccaacgacaccgatgtgttcgtgctgaacaacacccggcctccactcggcaattggttcggctgcacctggatgaactccacaggcttcaccaaagtgtgcggcgcacctccttgtggctcctctggatgtcctaccgactgttttcggaagcaccccgaagccacatactccagatgcggatctggcccctggatcaccccaagatgtatggtggattatccttatcggctgtggcattatccgtgcacgatcaactacaccatcttcaaagtgcggatgtacgtcggcggcgtggaacacagactggaagccgcctgtaattggaccagaggcgagagatgtgatctcgaggaccgggacagatccgagtga

SEQ ID NO: 21 - protein, 227 aa, Artificial; identical to SEQ ID NO: 11
  E2D123 amino acid sequence

SEQ ID NO: 22 - DNA, 1319 bp, Artificial
  F11-R-Flank sequence
ttaaccgagtttctgcattattgtaattcgtatgctggcaccatcaaagaatcacttctaaaagatatcaatatcacacatacaaatattactaccctattgaatgagacagccaaggttatcaagttagtaaaatctctggtagataaagaagatactgatattgtgaataatttcattaccaaagaaattaaaaacagagacaaaatagttaatagtttgtctctatcaaacctggactttcgtttgtaaattggggctttttgtacaataaatgggtgttgccaatgattcatcccctgaatatcaatggatgtctccccatagattatcagatactgttatattaggagactgtttgtattttaacaatataatgtcccaattagatttacaccaaaattgggctccatcagttagattgttaaattattttaagaattttaataaggaaacactactaaagatagaagagaatgattacattaattcatcctttttccaacaaaaggataaacgattttatcctataaacgacgatttttatcacatatctacaggaggatatggtatagtctttaagatagataactatgtagtaaaatttgtattcgaggccacaaaattatatagtcccatggaaactacggcggagttcacagtacccaaatttctatacaacaatctaaagggagatgaaaaaaaattaatcgtgtgtgcgtgggccatgggattaaactataaattaacatttttacatactctgtataaacgtgttcttcatatgttgctattattgatacaaactatggatggtcaggaactatcattgagatattcttctaaagtttttttaaaggcgtttaacgagagaaaggacagtatcaaattcgtgaaattactatcccacttttatccggcagttattaacagtaatattaatgttataaactattttaaccgcatgtttcactttttcgaacatgaaaagagaactaactacgaatacgaaagaggaaatattataatttttcccctagcactgtattcggcagataaagtagataccgagctagctatcaaattaggatttaaatctttggtacaatacataaagtttatctttttacagatggctctgttatacattaaaatttacgaactaccatgctgcgacaactttttacacgcagatcttaaacccgataatatcttactttttgattccaatgaaccaataataattcatctaaaggataaaaagtttgtttttaatgaacgtattaaatcggcattaaacgactttgacttttcccaag

(19) United States
(12) Patent Application Publication
(10) Pub. No.: US 2023/0181726 A1
(43) Pub. Date: Jun. 15, 2023

(54) A HEPATITIS C NUCLEIC ACID VACCINE COMPRISING A VARIABLE DOMAIN DELETED E2 POLYPEPTIDE

(71) Applicants: Macfarlane Burnet Institute for Medical Research and Public Health Limited, Melbourne, Victoria (AU); CHANCELLOR, MASTERS AND SCHOLARS OF THE UNIVERSITY OF OXFORD, Oxford (GB)

(72) Inventors: Heidi DRUMMER, Melbourne, Victoria (AU); Eleanor Jane BARNES, Oxford (GB); Senthil Kumar CHINNAKANNAN, Oxford (GB)

(73) Assignees: Macfarlane Burnet Institute for Medical Research and Public Health Limited, Melbourne, Victoria (AU); The Chancellor, Masters and Scholars of the University of Oxford, Oxford (GB)

(21) Appl. No.: 17/924,646

(22) PCT Filed: May 11, 2021
(86) PCT No.: PCT/AU2021/050437

§ 371 Date: Nov. 10, 2022

(30) Foreign Application Priority Data
May 11, 2020 (AU) 2020901506

(51) Int. Cl. A61K 39/29; A61P 31/14

(52) U.S. Cl. CPC A61K 39/29; A61P 31/14; A61K 2039/575

(57) ABSTRACT

A pharmaceutical composition comprising a nucleic acid molecule encoding a variable domain deleted E2 polypeptide of HCV (e.g., E2Delta123). The composition is suitable for use, for use, or when used, in the treatment or prevention of HCV infection. The nucleic acid molecule may be DNA or RNA or a modified or synthetic form, or contained within a plasmid, a viral or non-viral vector for vaccination, a polynucleotide expression cassette, or a cell for vector propagation. Methods of administration as prime and boost vaccinations are also provided.

### FIELD OF THE DISCLOSURE

This disclosure relates to treatment or prevention of hepatitis C virus (HCV) using an modified HCV E2 vaccine antigen in the form of a nucleic acid molecule encoding the antigen which is expressed in a subject and stimulates a functional and cross protective immune response to HCV.

### BACKGROUND ART

Hepatitis C virus is a small, enveloped positive sense RNA virus belongs to the unique genus hepacivirus within the flaviviridae family. HCV is classified into 7 genotypes (GT1-7) with genotypes 1-3 globally distributed. Infected individuals harbour a swarm of closely related viruses referred to as a quasispecies. This degree of sequence variation exceeds that observed for HIV and influenza, posing major challenges for vaccine development. HCV encodes two structural envelope glycoproteins, E1 and E2 that are cleaved from the viral polyprotein by signal peptidases. E1E2 functions as a heterodimer to mediate viral entry. E2 mediates attachment to the cellular receptors, CD81 and scavenger receptor class B type 1, via its receptor binding domain (RBD). The function of E1 is currently unknown.

The genome of HCV is a single-stranded, positive-sense RNA of approximately 9,600 nucleotides with only one large open reading frame (ORF) that encodes a single polyprotein of 3,010 to 3,033 amino acid residues. The HCV ORF is flanked by highly conserved 5′- and 3′-untranslated regions (5′-UTR and 3′-UTR) required for viral replication. The 5′-UTR forms extensive secondary structures and regulates translation initiation in an internal ribosome entry site (IRES)-dependent manner. However, in contrast to picornaviruses, the HCV IRES can bind the 40S ribosomal subunit directly in the absence of any other canonical translation initiation factor, thereby positioning the authentic initiator AUG codon of the ORF precisely at the P site of the ribosome. The 3′-UTR lacks a poly(A) tail and is composed of three sequence elements supposed to be involved in RNA replication: a nonconserved variable region (30 to 50 nucleotides), a poly(U · C) stretch (20 to 200 nucleotides), and a conserved 98-nucleotide sequence, termed the 3′ X region, which forms a three stem-loop (SL) structure.

Hepatitis C virus affects more than 70 million people and causes more than 700,000 deaths each year. It is estimated that 35 million people have undiagnosed infections providing a means for continued viral spread. Acute hepatitis C is marked by appearance of HCV RNA in serum within 1 to 2 weeks of exposure followed by serum alanine aminotransferase (ALT) elevations, and then symptoms of jaundice. Antibody to HCV (anti-HCV) tends to arise late. In acute resolving hepatitis, HCV RNA is cleared and serum ALT levels fall to normal. However, 55% to 85% of patients do not clear virus, and develop chronic hepatitis C. Chronic hepatitis C is often asymptomatic, but is usually associated with persistent or fluctuating elevations in ALT levels. The chronic sequelae of hepatitis C include progressive hepatic fibrosis, cirrhosis, and hepatocellular carcinoma. Extra-hepatic manifestations include the autoimmune disease, sicca syndrome, cryoglobulinemia, glomerulonephritis, and porphyria cutanea tarda. Direct acting antivirals can cure HCV infections in more than 90% of cases however, alone antivirals are unlikely to achieve HCV elimination because of their high cost, the risk of reinfection, and the fact that most people who are infected are not diagnosed with HCV. It is argued that a vaccine is needed that limits transmission in order to achieve a significant reduction in HCV infections.

Central to the potential success of an HCV vaccine is the ability to confer broad and effective protection against the seven circulating HCV genotypes. Each genotype displays -30% variation at the protein level, and comprises over 67 subtypes which themselves display -20% variation at the protein level. Recent studies have shown that humans who clear their infection spontaneously generate broadly neutralizing antibodies (bNAbs) early in infection, and passive immunization of animals with human bNAbs can protect them from HCV challenge.

The neutralizing antibody response is predominantly directed towards the receptor binding domain (RBD) of E2 viral envelope glycoprotein (Drummer et al., Microbiol. 5:329, 2014). However, HCV E2 displays multiple immune evasion mechanisms to restrict the generation of bNAbs including for example, glycan shielding, focussed amino acid sequence evolution in hypervariable regions (HVRs) that allosterically suppress the presentation of bNAb epitopes, and immunodominance of epitopes that preferentially generate isolate-specific NAbs that drive immune escape and non-neutralizing antibodies (Drummer et al. supra).

As determined previously by the inventor, in native HCV E2 forms, the hypervariable regions act in concert to occlude conserved epitopes that determine broadly neutralising antibodies (bNAb) and this prevents the generation of bNAbs. Instead, isolate-specific epitopes in the variable regions and non-neutralizing epitopes dominate the antibody response. As described in WO 2008/022401 and WO 2012/0168637 the inventor/s has/have developed modified forms of HCV E2 and in particular, one in which the three surface-exposed HVRs are deleted from the RBD (referred to as Delta123E2₆₆₁). This HVR depleted form of E2 was compared to wild type E2 and compared to different presentations of the molecule including monomeric, dimeric, trimeric and higher repetitive forms. As reported in Hepatology 65(4), 2017 the high molecular weight, disulphide-linked oligomers of E2 lacking the three hypervariable regions (D123HMW) elicit high-titre bNAbs able to neutralize all seven genotypes of HCV in experimental animals. The HMW oligomeric form of E2 was far more effective than monomeric or dimeric forms of E2 which even when HVR depleted stimulate non-neutralizing antibodies. Prior to this work it was assumed that the monomeric form of HCV E2 would provide the optimal vaccine construct. The nature of the high molecular weight species identified for example in mammalian expression cell culture were poorly characterised and their significance for a vaccine was not understood.

However, there are problems in producing D123HMW E2 (high molecular weight oligomeric form of E2 lacking hypervariable regions HVR1, HVR2 and igVR) in a form suitable for use as a vaccine because it is produced spontaneously during mammalian cell expression at very low yield. In fact the inventor/s have determined that E2 is produced during mammalian cell expression in multiple heterogeneous forms, including monomers, dimers, oligomers and other conformations. Thus a complex isolation procedure would be required to separate the D123HMW E2 from the other undesirable forms of E2. Another option would be to try to simplify in vitro protein production of homogeneous HMW D123 HCV E2 for human trials. One surprisingly effective approach described by the inventor/s (see International Publication No. 2018/058177 and based on an invention described in International Publication No. WO 2012/016290) and has been to produce a cysteine modified form of HCV E2 D123 polypeptide which is expressed almost entirely as monomeric E2 D123 and then processing the monomeric HCV E2 in vitro to form a homogenous HMW E2 polypeptide suitable for vaccination studies.

Replication defective adenoviral vectors have previously been shown to prime sustained T-cells responses to non-structural (NS) HCV proteins. For vaccination in man, rare (low seroprevalence) adenoviral or chimpanzee adenoviral vectors are employed to avoid pre-existing anti-vector immune responses that might interfere with vaccine efficiacy. Boosting regimen, if required to boost the T-cell response are proposed to comprise different vectors such as a modified poxviral vector.

Given the global problem of epidemic HCV infections, especially in developing countries, there is an urgent unmet need for an effective HCV vaccine and one that elicits at least high levels of functional neutralizing antibodies and low levels of non-functional antibodies.

### SUMMARY OF THE DISCLOSURE

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the singular forms “a”, “an” and “the” include plural aspects unless the context clearly dictates otherwise. Thus, for example, reference to “a composition” includes a single composition, as well as two or more compositions; reference to “an agent” includes one agent, as well as two or more agents; reference to “the disclosure” includes single and multiple aspects of the disclosure and so forth.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/- 10%, or +/-5%, of the designated value.

The term “variable region deleted” includes 1, 2, or 3 of the variable regions of HCV E2 deleted or substituted. Although exemplified by deleting the variable domains, the skilled person will appreciate that substitution of the regions or mutations could achieve the same result as a deletion.

A high level of human intervention was required to modify the native HCV E2 polypeptide to produce the high molecular weight (HMW or higher order >3/4/5) variable region deleted (e.g., Delta123) HCV E2 polypeptide as a homogeneous vaccine candidate able to engender effective functional antibodies and low levels of non-functional antibodies. There would have been no expectation in the art that a nucleic acid molecule encoding the Delta123 E2 monomer would, upon expression, behave similarly to the HMW Delta123 E2, and preferentially engender functional antibodies over non-functional antibodies upon administration to a mammalian host. Recombinant HCV E2 glycoproteins expressed by eukaryotic cell lines in vitro are known to be generally present as mixtures of monomers, dimers, trimers and higher order forms. The presence of monomeric forms of antigen would have been routinely expected to elicit high levels of non-functional antibodies at the expense of functional antibody production. In accordance with the present disclosure, the inventors have determined that neutralization titres after administration of the encoding nucleic acid form of Delta123E2 are surprisingly higher and the neutralising antibodies are more cross reactive than when the Delta123HMW E2 polypeptide is administered in proteinaceous form alone. Accordingly, it is proposed to administer an HCV E2 vaccine in nucleic acid form, such as in a viral or non-viral vector which provides therefore a viable, low cost yet effective alternative to the administration of protein alone.

In one aspect therefore the present invention provides a pharmaceutical composition comprising a viral expression vector encoding a variable domain deleted E2 polypeptide of HCV, such as Delta123E2. In one embodiment, the viral vector is an adenoviral expression vector, including a synthetic version thereof. In one embodiment, the viral vector is a poxviral or vaccinia-based expression vector, such as an attenuated vaccinia genome vector, including a synthetic versions and precursors thereof. Illustrative expression vectors and expression cassettes are represented in the Figures such as FIG. 20 and FIGS. 27 to 35, but many versions or alternative elements (shuttle plasmids, leader sequence, promoters, polyadenylation sequence, drug resistance operator sites, BAC genomes, and restriction sites) are known in the art. Viral rescue and propagation in suitable cell lines are also known in the art.

In one embodiment, the viral vector is an adenoviral genome based viral vector.

In one embodiment, the viral vector is poxviral or vaccinia genome based viral vector.

In the embodiment, the present invention provides cells of a cell line expressing a viral vector encoding a variable domain deleted E2 polypeptide as described herein. Thus in one embodiment, the viral vector may be an attenuated adenoviral vector or an attenuated vaccinia vector.

In one embodiment, the present invention provides for use of a vector as described herein, or a cell expressing a viral vector encoding a variable domain deleted E2 polypeptide as described herein in the manufacture of a nucleic acid/viral vector based vaccine or immunogenic composition or pharmaceutical or physiological composition comprising same.

In one aspect, this disclosure provides a method of treatment or prophylaxis of HCV infection in a subject comprising administering to the subject a nucleic acid molecule encoding a variable domain deleted HCV E2 proteinaceous molecule, such as Delta123E2 or a variant thereof.

In one aspect, therefore, this disclosure provides a physiological or pharmaceutical composition conveniently prepared according to known pharmaceutical manufacturing techniques. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g., without limitation, intravenous, intranasal, intradermal, oral or parenteral for use in therapy or for in the treatment or prevention of HCV infection, wherein the nucleic acid is administered to a subject and wherein the variable domain deleted E2 protein is produced and generates an immune response including a functional B-cell response to HCV in the subject. Compositions include physiologically or pharmacologically or pharmaceutically acceptable vehicles that are not biologically or otherwise undesirable. Pharmacologically acceptable salts, esters, pro-drugs, or derivatives of a compound described here is a salt, ester, pro-drug, or derivative that is not biologically or otherwise undesirable.

Vectors and expression cassettes are suitable for or adapted for expression of variable domain deleted E2 in endogenous human cells i.e., for human treatment or vaccine recipients.

In one embodiment the composition is for use in therapy as a vaccine.

In one embodiment, the present invention enables a composition, including a pharmaceutical composition, comprising a nucleic acid molecule encoding a soluble variable domain deleted E2 polypeptide of HCV for use, or when used, in the treatment or prevention of HCV infection, wherein the use comprises administering the nucleic acid to a subject and wherein the variable domain deleted E2 protein is produced in the subject and generates an immune response including a functional B-cell response to HCV in the subject.

In this aspect, the use may be for use in enhancing the neutralising B-cell response wherein the nucleic acid molecule, when expressed, generates in the subject an enhanced B-cell response compared to that generated using HMW Delta123E2.

While exemplified with Delta123, it is envisaged that Delta 23 would also effect more neutralising cross reactive B-cell responses than wild type E2.

In one embodiment, the nucleic acid administration comprises priming with a viral vector encoding variable domain deleted E2 and boosting with the same or a heterologous vector or nucleic acid encoding variable domain deleted E2, or a high molecular weight variable domain deleted E2 polypeptide (eg. HMWDelta123).

In one embodiment the nucleic acid molecule is expressed by a viral vector such as ChadOX1 or MVA as set out in FIG. 20 and FIGS. 27 to 35.

In one embodiment, the nucleic acid molecule further engenders a T-cell response to the encoded glycoprotein.

A “functional” B-cell response to HCV as used herein means the production of cross reactive neutralising antibodies able to reduce host cell invasion by HCV viruses and a low or no production of non-neutralising antibodies. A primarily non-functional B-cell response means the production of antibodies that do not recognise the RBD of HCV or do not have cross-neutralizing potential. For example, monomeric wild type E2 polypeptide when administered, elicits low levels of antibodies that recognise the core domain with cross-neutralising potential and high levels of antibodies that are non-neutralising and poorly cross reactive with other variants

The terms “encoding” and “capable of expressing” are used herein interchangeably.

In one embodiment, the nucleic acid molecule encoding a variable domain deleted E2 of HCV is contained within or attached to a viral or non-viral vector for administration and/or intracellular delivery.

In one embodiment, the nucleic acid molecule encoding a variable domain deleted E2 of HCV is RNA or DNA or RNA:DNA hybrid or a modified or synthetic form thereof.

In one embodiment, the vector is a viral vector or a non-viral vector. Viruses, which are useful as vectors include, but are not limited to, retroviruses, adenoviruses, adeno-associated viruses, herpes viruses and lentiviruses, and attenuated forms thereof, each of which have their own advantages and disadvantages as known in the art. Viral vectors specifically include without limitation an adenoviral vector and a poxviral vector.

Non-viral vectors or attachments/conjugates include lipids, carbohydrates, proteins, peptides, nanoparticles, liposomes, viral-like particles, virosomes, emulsions. Amphipathic agents such as lipids may exist in aggregates as micelles, insoluble monolayers, liquid crystals or lamellar layers in aqueous solution.

In one embodiment, the nucleic acid molecule encodes a variable domain deleted E2 of HCV that has all three surface exposed variable domains deleted (e.g., Delta123E2). Other variants could include 1 or 2 variable domains deleted. In one embodiment HVR1 (394-408) is not deleted. In one embodiment HVR2 (460-495) and igVR (570-580) regions are substantially or entirely deleted.

In one embodiment, the nucleic acid molecules described herein do not encode an HCV genome or full length E2 polypeptide not having variable domain deletions.

In one non-limiting embodiment, the nucleic acid molecule encodes HCV E2 polypeptide having the amino acid sequence set out in SEQ ID NO:1 which is the protein sequence of Delta123 E2 employed in the examples.

In another embodiment, a cysteine modified version of the receptor binding domain as described by the inventor in WO 2012/016290 is enabled. Thus versions of SEQ ID NO: 1 or 2 are contemplated in which at least four mutated or disrupted cysteines selected from C581 to C620 are encoded and the encoded polypeptide retains CD81 binding.

Cysteine modified versions of E2 as described in WO 2012/016290 are expressed recombinantly as monomers and may be re-folded into high molecular weight forms.

In one embodiment, the nucleic acid molecule comprises the HCV-based sequence set out in SEQ ID NO:2 which is the nucleotide sequence of Delta123E2 codon optimised for human expression. In one embodiment, the nucleic acid molecule does not encode any other envelope proteins. In one embodiment, the nucleic acid molecule comprises the sequence set out in SEQ ID NO:2 or the corresponding sequence from any genotype of HCV, such as G2, G3, G4, G5, G6 or G7. Variants of SEQ ID NO: 2 having at least 80% sequence identity are contemplated, including 81%, 82%, 83%, 84%, 85%, 86%, 87%, 88%, 89%, 90%, 91%, 92%, 93%, 94%, 95%, 96%, 97%, 98% or 99% sequence identity.

Reference to variants includes parts, derivatives, and chemical analogs. Chemical analogs contemplated include modification of side chains, incorporation of unnatural amino acids and/or their derivatives during synthesis and the use of linkers or crosslinkers or other methods to inter alia impose conformational constraints.

Reference to higher or lower levels means a significantly higher or lower levels e.g., at least 50%, 75%, 100% , 3-fold, 4-fold, 5-fold or more or less antibody or isotype compared to an appropriate control. Total antibody titres, isotypes and neutralising antibody titres are measured by art recognised techniques such as those described in the examples.

In one embodiment, the functional B cell response includes a total antibody titre that is lower than the total antibody titre generated after corresponding administration of high molecular weight variable domain deleted E2 polypeptide (HMWDelta123).

In one embodiment, the functional B-cell response is comparable to the functional B-cell response generated after corresponding administration of high molecular weight variable domain deleted E2 polypeptide (HMWDelta123).

In some embodiments, the functional immune response comprises CD81 inhibition titres comparable to or higher than the CD81 inhibition titres generated after corresponding administration of high molecular weight variable domain deleted E2 polypeptide (HMWDelta123)

In one embodiment, the composition comprises another physiologically, therapeutically or prophylactically active ingredient.

Accordingly, the present disclosure enables the use of a nucleic acid molecule encoding a variable domain deleted E2 polypeptide of HCV in, or in the manufacture of a NA or NA-vector medicament for the treatment or prevention of HCV infection in a subject.

In one embodiment, a B-cell response primed by the viral vector expressing the variable domain delta E2, enhances the immune response to HCV.

In one embodiment, the nucleic acid administered E2, enhances the immune response to HCV above that provided by the HMW Delta123E2.

As used herein “subjects” contemplated in the present description are humans or animals including laboratory or art-accepted test or vehicle animals. In an embodiment, the subject is a mammal. Preferably, the subject is a human, however the present description extends to treatment and/or prophylaxis of other mammalian patients including primates and laboratory test animals (e.g. mice, rabbits, rats, guinea pigs).

In some embodiments, the nucleic acid molecule encoding a variable domain deleted E2 polypeptide of HCV is contained within a viral or non-viral vector for vaccination.

In one embodiment, the nucleic acid molecule encoding a variable domain deleted E2 of HCV is RNA or DNA or a modified form thereof.

In one embodiment, the nucleic acid sequence is codon optimised for expression in human cells.

In one embodiment, the viral vector is an adenoviral vector. In one embodiment the viral vector is poxviral vector, such as MVA.

In one embodiment, the nucleic acid molecule encodes a variable domain deleted E2 of HCV that has all three surface exposed variable domains deleted (Delta123E2). In one embodiment, the nucleic acid molecule encodes an HCV E2 polypeptide having the amino acid sequence set out in SEQ ID NO: 1 or the corresponding sequence from a strain of HCV genotype 2 to 7. In an illustrative embodiment, the nucleic acid molecule comprises the sequence set out in SEQ ID NO:2 or a sequence having at least 80% identity thereto.

The present disclosure also enables a method of treatment or prevention of HCV infection, comprising the administration of a composition comprising a nucleic acid molecule encoding a variable domain deleted E2 polypeptide of HCV to a subject for a time and under conditions to generate a functional B-cell response to HCV in the subject.

The present disclosure also enables a method of inducing a functional B-cell response to HCV in a subject, comprising the administration of a composition comprising a nucleic acid molecule encoding a variable domain deleted E2 polypeptide of HCV to a subject for a time and under conditions to generate a functional B-cell response to HCV in the subject.

In one embodiment, the adenoviral genome based viral vector comprises one or more of, the polynucleotide sequence of ChAdOx1 set out in FIG. 27, the polynucleotide sequence of ChAdOx1-TPA-E2D123 set out in FIG. 27, the vector as arranged in FIG. 28, the polynucleotide sequence set out in FIG. 29, the immunogen cassette lay out of FIG. 30, one or two or more of the immunogen cassette sequences set out in FIG. 31, the polynucleotide sequence of FIG. 32.

In one embodiment, the vaccinia genome based viral vector comprises one or more of, the polynucleotide sequence of MVA set out in FIG. 33, the polynucleotide sequence of MVA-TPA-E2Delta123 set out in FIG. 33, the vector layout of FIG. 34 or one or two or more of the elements by sequence set out in FIG. 35.

In one embodiment, a Chimpanzee adenovirus vector encoding E2 D123 sequence is administered at day 0 (d0) optionally followed by a one or more boosts of same over the following 1 to 6 months.

In one embodiment, a Chimpanzee adenovirus vector encoding the Genotype 1a E2 D123 sequence is administered at day 0 (d0) optionally followed by a one or more boosts of same over the following 1 to 6 months.

In one embodiment, a Chimpanzee adenovirus vector encoding E2 D123 sequence is administered at day 0 (d0) optionally followed by a one or more boosts over the following 1 to 6 months.

In one embodiment, a Chimpanzee adenovirus vector encoding the Genotype 1a E2 D123 sequence is administered at day 0 (d0) optionally followed by a one or more boosts over the following 1 to 6 months.

In one embodiment, boost administration may be of a composition comprising soluble purified high molecular weight E2 D123 protein.

In one embodiment, boost administration may be of a composition comprising a heterologous (non-adenoviral) viral vector encoding E2 D123 protein.

In one embodiment, the heterologous viral vector is a chordopox genome based viral vector, such as a vaccinia genome based viral vector.

In one embodiment, boosting is with soluble purified high molecular weight E2 D123 protein.

In one embodiment of the method, the composition comprising nucleic acid encoding HCV E2 Delta 123 is administered as a prime vaccination, or as a prime and a boost vaccination.

In one embodiment, the composition is administered as a prime vaccination, followed by two booster vaccinations.

In one embodiment, the prime vaccination is with MVA and the booster vaccination is with same or with a composition comprising soluble high molecular weight variable domain deleted E2 protein.

In one embodiment, the prime vaccination is with MVA and the booster vaccination is with same and/or with a composition comprising soluble high molecular weight variable domain deleted E2 protein.

In one embodiment, the prime vaccination is with MVA and the booster vaccination is with same and/or with soluble purified high molecular weight variable domain deleted E2 protein.

In one embodiment, the prime vaccination is with MVA and the booster vaccination is with same or with soluble purified high molecular weight variable domain deleted E2 protein.

In one embodiment, the prime vaccination is with MVA and the booster vaccination is with same or with soluble purified high molecular weight E2 D123 protein.

In one embodiment, the composition is administered as a prime vaccination and wherein a booster vaccination comprises high molecular weight variable domain deleted E2 polypeptide of HCV.

### BRIEF DESCRIPTION OF THE DRAWINGS

FIG. 1 shows the schematic representation of immunization experiment carried out in C57B16 mice comparing immunogenicity of D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3).

FIG. 2 shows the reciprocal antibody titres generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3) at all time points graphed by experiment group. * p<0.0332, ** p<0.0021, *** p<0.002, **** p<0.0001.

FIG. 3 shows the reciprocal antibody titres generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3) at all time points graphed by vaccine schedule. * p<0.0332, ** p<0.0021, *** p<0.002, **** p<0.0001.

FIG. 4 shows the antibody response curves at day 56 generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3) graphed by experimental group.

FIG. 5 shows the antibody titres at day 56 generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3) graphed by experimental group. * p<0.0332, ** p<0.0021, *** p<0.002, **** p<0.0001.

FIG. 6 shows the ability of immune sera generated at day 56 to inhibit the interaction between HCV cellular receptor CD81 and the E2 receptor binding domain. The ability of the immune serum to inhibit 50% (A) or 80% (B) of E2 binding CD81 was calculated from 12 point dilution curves. * p<0.0332, ** p<0.0021, *** p<0.002, **** p<0.0001.

FIG. 7 shows the antibody titres at day 56 towards the 408-428 region of HCV E2 generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). * p<0.0332, ** p<0.0021, *** p<0.002, **** p<0.0001.

FIG. 8 shows the antibody titres at day 56 towards the 430-451 region of HCV E2 generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). * p<0.0332, ** p<0.0021, *** p<0.002, **** p<0.0001.

FIG. 9 shows the antibody titres at day 56 towards the 523-549 region of HCV E2 generated in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). * p<0.0332, ** p<0.0021, *** p<0.002, **** p<0.0001.

FIG. 10 shows the homologous neutralization titres at day 56 in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). * p<0.0332, ** p<0.0021, *** p<0.002, **** p<0.0001.

FIG. 11 shows the heterologous neutralization titres at day 56 in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3) towards genotype 3a HCV.

FIG. 12 shows the heterologous neutralization titres at day 56 in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3) towards genotype 5a HCV.

FIG. 13 shows the isotypes of antibodies generated at day 56 in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). Isotypes were measured using a mouse isotyping kit and expressed as a percentage of the total antibody isotypes observed.

FIG. 14 shows the isotypes of antibodies generated at day 56 in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). Isotypes were measured using a mouse isotyping kit and expressed as a percentage of the total antibody isotypes observed in a pie chart. This is the same data as shown in FIG. 13 presented in a pie chart.

FIG. 15 compares the isotypes of antibodies generated at day 56 in animals vaccinated with D123 encoded within Chimpanzee adenovirus 1 (Group 1) and boosted with same, or as a high molecular weight soluble protein and boosted with same (Group 2), or a prime with D123 encoded within Chimpanzee adenovirus 1 and boosted with high molecular weight soluble protein (Group 3). Isotypes were measured using a mouse isotyping kit and expressed as a percentage of the total antibody isotypes. * p<0.033, ** p<0.002, *** p<0.001.

FIG. 16 shows the protein and DNA sequences of D123 used in this study for the purpose of illustration only.

FIG. 17 shows the cross-reactive antibody titres at day 56 towards the genotype 3a (S52 isolate) the 408-428 epitope I region, 430-451 epitope II region, and 523-549 CD81 binding loop region (epitope III) of HCV E2 generated in animals vaccinated with D123 encoded within Chimpanzee adenovirus (ChAd or ChAdOx1) (Group 1) and boosted with same, or as a high molecular weight soluble protein and boosted with same (Group 2), or a prime with D123 encoded within Chimpanzee adenovirus and boosted with high molecular weight soluble protein (Group 3). * p<0.0332, ** p<0.0021, *** p<0.002, **** p<0.0001.

FIG. 18 shows the IgG2a titre of immune sera calculated as a function of the IgG1 titre and displayed as IgG1 log10 titre divided by IgG2a log10 titre (IgG1/IgG2a). The lower the titre, the closer the IgG2a to IgG1 ratio is to 1:1, indicative of IgG1 to IgG2a class switching. The reciprocal titre (1/IgG1:IgG2a) was plotted against the HCVpp ID50 titre for immune sera from animals that received C/P/P and P/P/P to determine any correlation. All bars are medians and interquartile ranges are displayed. The D′Agostino and Pearson test was used to determine normality of data distribution and Kruskal-Wallis with multiple comparisons were performed to determine significant differences between two group medians at a 95% confidence interval. P values indicate significant difference between groups when <0.05*, <0.01**, <0.001***, <0.0001****. When the IgG2a was expressed as a function of the IgG1 titre (IgG1/IgG2a), ChAd-E2A123 immune sera (groups 1 and 3) had a significantly lower reciprocal titre compared to E2Δ123_{HMW} immune sera indicative of increased GC class switching (p<0.0001**** for group-1 v. group-2, p=0.0014** for group-2 v. group-3). For E2Δ123_{HMW} immune sera (group-2 and -3 combined), the IgG1:IgG2a reciprocal titre positively correlated with HCVpp neutralising titres (r=0.3974, p=0.0492).

FIG. 19 shows the reciprocal ID50 inhibitory titre of immune sera as a function of the overall Ab titre, denoted functional antibody index. The functional antibody index was calculated for both E2-CD81 inhibition shown as CD81 blockade. The functional antibody index was calculated for inhibition of virus entry of homologous pseudotyped virus shown as neutralization. When the reciprocal ID50 titres (CD81 inhibition and HCVpp) are interpreted in relationship to the overall Ab titre (functional antibody index), the ChAd-E2Δ123 immune sera (group-1 and -3) had a significantly higher functional index compared to E2Δ123_{HMW} immune sera indicating a greater neutralising capacity of the vaccine-induced Ab response relative to the total Ab titre.

FIG. 20 shows the schematic of the viral vectors used to drive expression of E2 D123. A. Schematic of the expression cassette for D123 in ChAdOx1. B. Schematic of the expression cassette for RBD in ChAdOx1 . C. Schematic of the expression cassette for D123 in the MVA.

FIG. 21 shows the immunization schedule for animals receiving ChAd-E2A123 prime followed by a MVA-E2Δ123 boost at week 4. Animals were bled two weeks after the boost and antibody and T cell reactivity measured. Animals received a week-0 ChAd-E2D123 prime (10⁸ infectious units [IU] in 40uL sterile PBS) followed by a week-4 MVA-E2D123 (10⁷ plaque forming units [PFU] in 40uL sterile PBS ).

FIG. 22A shows the reactivity of immune serum raised to immunization with ChAd-E2Δ123/MVA-E2Δ123 as shown in FIG. 21. Immune serum was assessed in ELISA against E2Δ123 monomer. Serial dilutions of immune serum was applied to E2Δ123 monomer coated plates and bound antibody detected with anti-mouse immunoglobulins conjugated to horse radish peroxidase and TMD substrate. The reciprocal titre required to achieve 10 times background binding was calculated. The results show that a ChAd-E2Δ123/MVA-E2Δ123 generates high titre antibodies reactive to E2. FIG. 22B shows the ability of immune sera generated at day 56 to inhibit the interaction between HCV cellular receptor CD81 and the E2 receptor binding domain. The ability of the immune serum to inhibit 50% of E2 binding CD81 was calculated from serial dilution curves. The results show that a ChAd-E2Δ123/MVA-E2Δ123 generates high titre antibodies capable of inhibiting the binding between homologous E2 and CD81.

FIG. 23A shows the reactivity of immune serum raised to immunization with ChAd-E2Δ123/MVA-E2Δ123 as shown in FIG. 21 to AS412 (E2₄₀₈₋₄₂₈), AS434 (E2₄₃₀₋₄₅₁), CD81 binding loop (E2₅₂₃₋₅₄₉). The reciprocal titre required to achieve 10 times background binding was calculated from serial dilution curves. The results show that all animals generated antibodies capable of binding to three different epitopes that are targets of broadly neutralizing antibodies. FIG. 23B shows the homologous (G1a) and heterologous (G3a) neutralization titres of immune serum raised ChAd-E2Δ123/MVA-E2Δ123 (FIG. 21). The reciprocal titre of immune serum required to inhibit 50% virus entry was was calculated from serial dilution curves. The results show that 7/10 animals generated antibodies capable of preventing entry of homologous genotype 1a hepatitis C virus into a Huh7 cell line and 6/10 animals generated antibodies capable of preventing entry of a heterologous genotype 3a virus into a Huh7 liver cell line. The limit of detection is shown by a dotted line.

FIG. 24 shows the T cell responses in mice immunized with ChAd-E2Δ123/MVA-E2Δ123 as shown in FIG. 21. Splenocytes were harvested at week-6 and stimulated ex vivo using HCV peptides (15mer overlapping by 11aa) covering the length of the HCV proteome (A and C) and for peptide pools (B and D): Core-E1-E2, NS3-4, and NS5, for gt-1a (H77), -1b (J4), or -3a (k3a650). IFNg⁺ CD4⁺ (A and B) and IFNg⁺ CD8⁺ (C and D) T-cell frequencies as a percentage of total CD4⁺ or CD8⁺ T cell frequencies, respectively, were determined via intracellular cytokine staining and flow cytometry. All data are plotted as medians and interquartile ranges. The results show that animals generated both CD4+ and CD8+ T cells towards the homologous peptides, focussed on epitopes within the coreE1E2 region, likely E2. While 2/7 animals tested possessed cross reactive CD4+ T cell responses towards genotype 1b, no CD8+ cross reactivity was observed.

FIG. 25 shows the E2 specific T cell responses in mice immunized with ChAd-E2Δ123/MVA-E2Δ123 as shown in FIG. 21. Splenocytes were harvested at week-6 and stimulated ex vivo using E2 peptide pool from genotype 1a (15mer overlapping by 11aa) covering the length of the E2 region. The data show that mice vaccinated with ChAd-E2Δ123/MVA-E2Δ123 generate E2 specific IFNg+ T cells.

FIG. 26 shows the analysis of polyfunctional T cell responses in mice immunized with ChAd-E2Δ123/MVA-E2Δ123 as shown in FIG. 21. Vaccine-induced T-cell polyfunctionality was determined via ICS and flow cytometry after splenocyte stimulation ex vivo using HCV peptides (15mer overlapping by 11aa) covering the length of the HCV proteome for gt-1a (H77) to detect produced cytokines, IFNg, TNFa, and IL-2. Pie bases are medians and calculated using Pestle and SPICE software. All data are plotted as medians and interquartile ranges. The results show that vaccination with ChAd-E2Δ123/MVA-E2Δ123 generates highly polyfunctional CD4+ and CD8+ T cell responses.

FIG. 27 provides an illustrative nucleotide sequence for ChAdOx1-TPA-E2D123.

FIG. 28 provides a layout map for ChAdOx1-TPA-E2D123.

FIG. 29 provides ChAdOx1 polynucleotide sequence 5′ to the TPA-E2D123 immunogen cassette.

FIG. 30 provides the TPA-E2D123 immunogen cassette layout.

FIG. 31 provides the TPA-E2D123 immunogen cassette sequences; CMV promoter with Tet Operator sequence; Extra sequence; Kozak Sequence; TPA sequence; TPA amino acid sequence; E2D123 sequence; E2D123 amino acid sequence; Extra sequence; Bovine Growth Hormone (BGH) polyA sequence.

FIG. 32 provides polynucleotide sequence of ChAdOx1 sequence 5′ to the TPA-E2D123 immunogen cassette.

FIG. 33 provides the polynucleotide sequence for MVA-TPA-E2D123.

FIG. 34 provides the layout for MVA-TPA-E2D123.

FIG. 35 provides sequence information for MVA-TTPA-E2D123; F11-L-Flank sequence; mH5 promoter sequence; TPA sequence; TPA amino acid sequence; E2D123 sequence; and F11-R-Flank sequence.

### KEY TO SEQUENCE LISTING

SEQ ID NO:1 D123 protein sequence including Trypsin leader sequence.

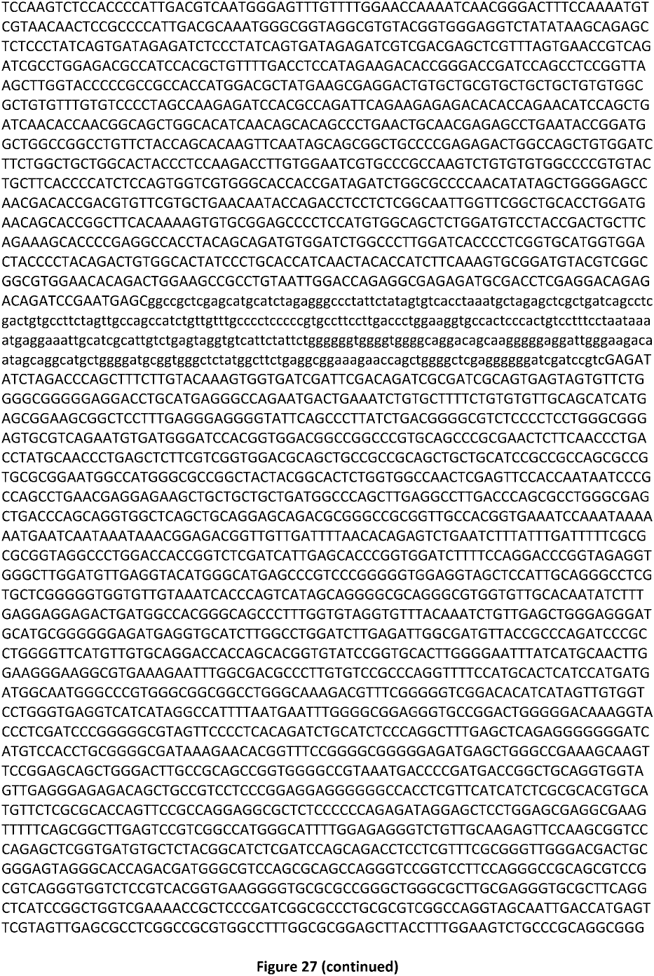

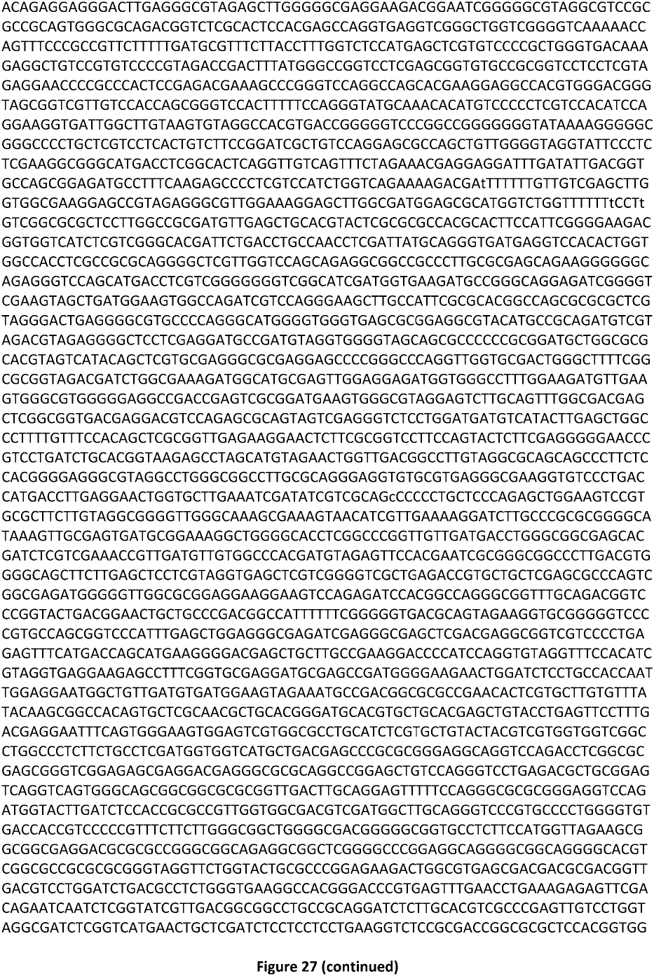

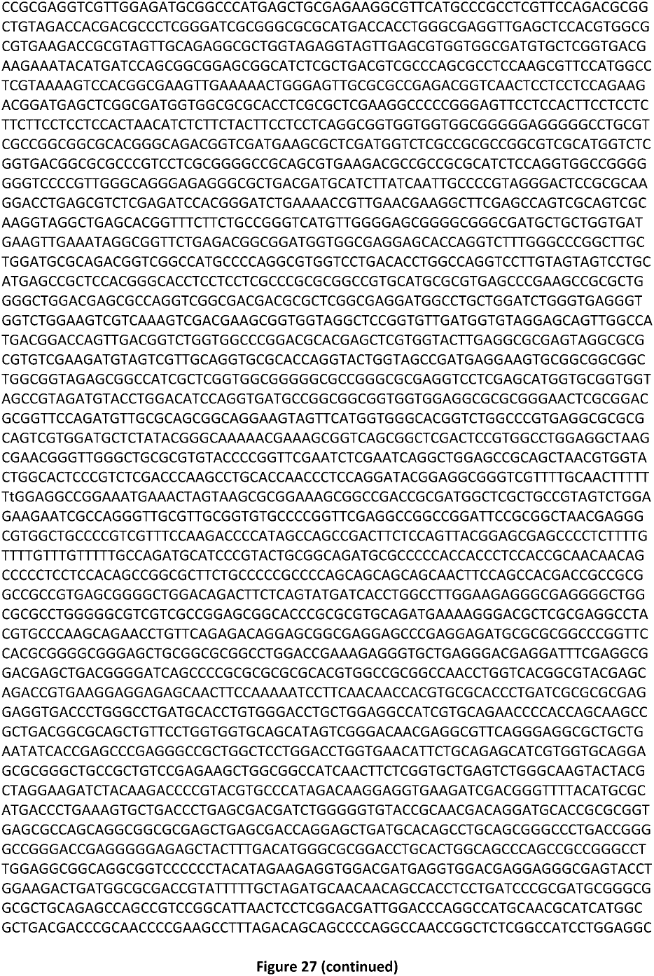

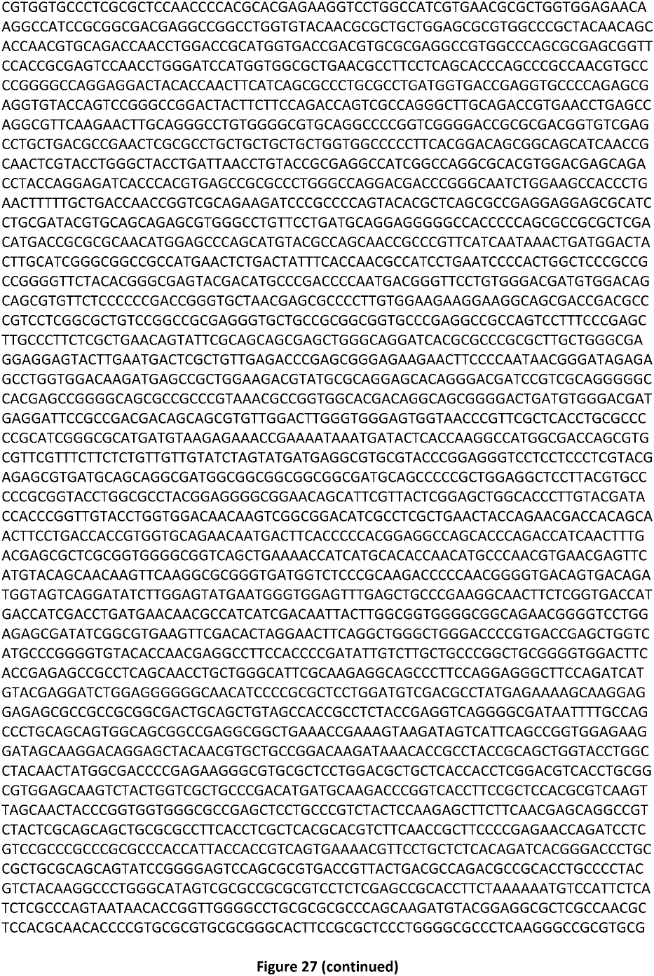

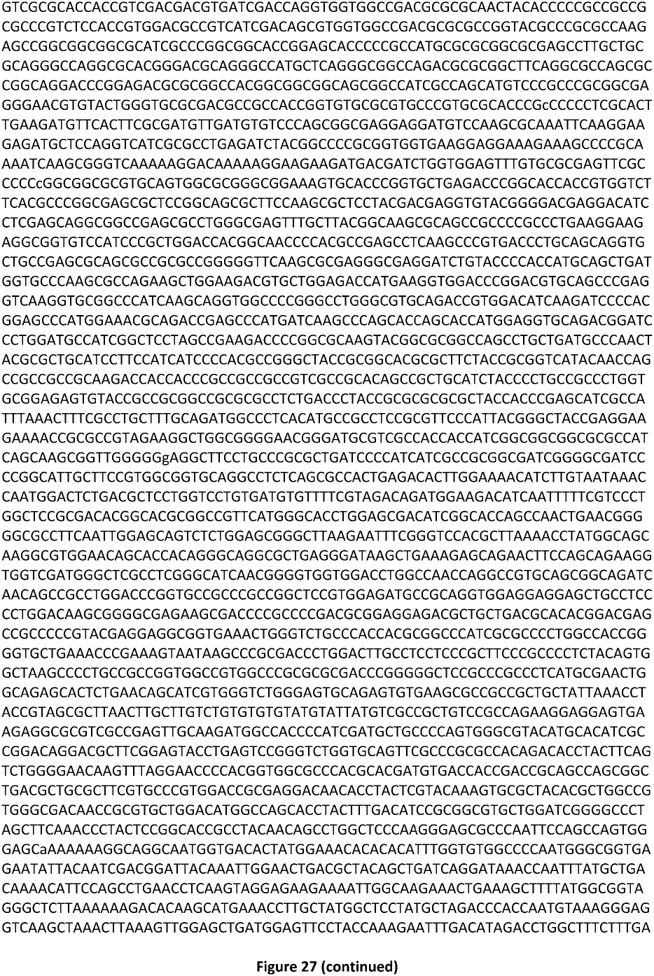

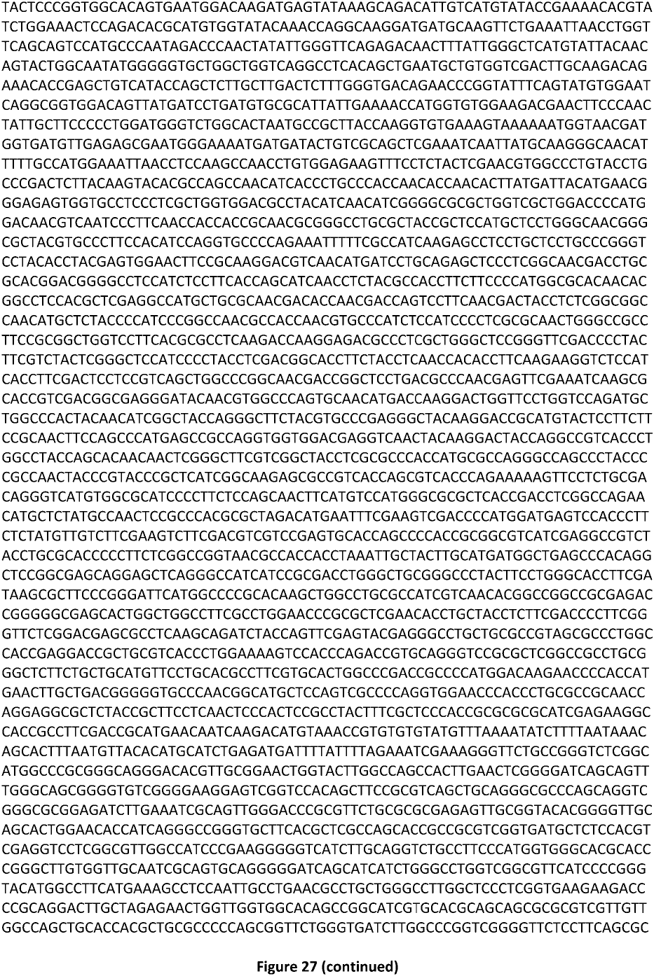

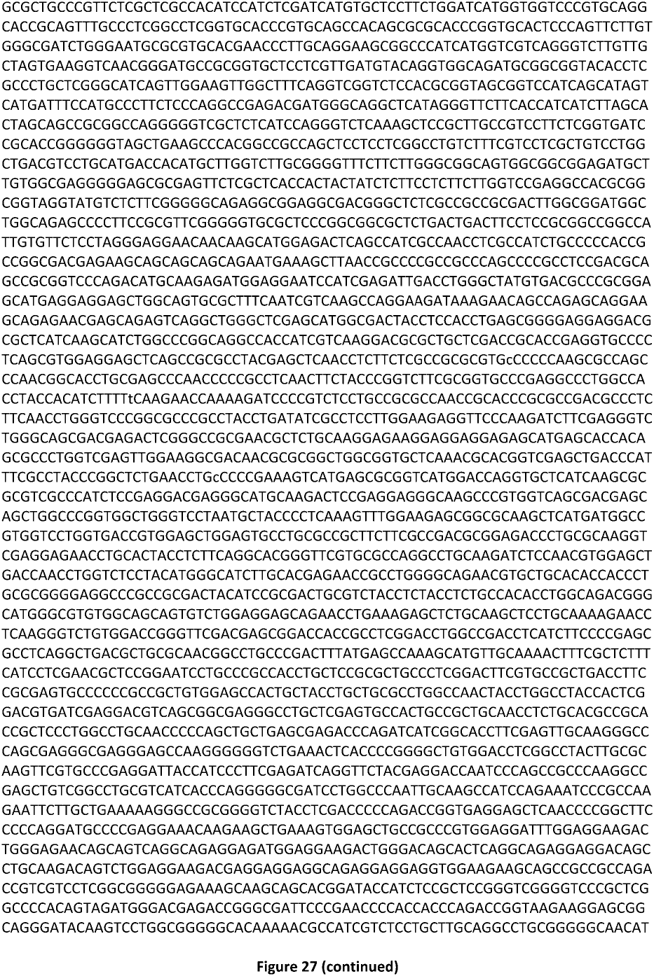

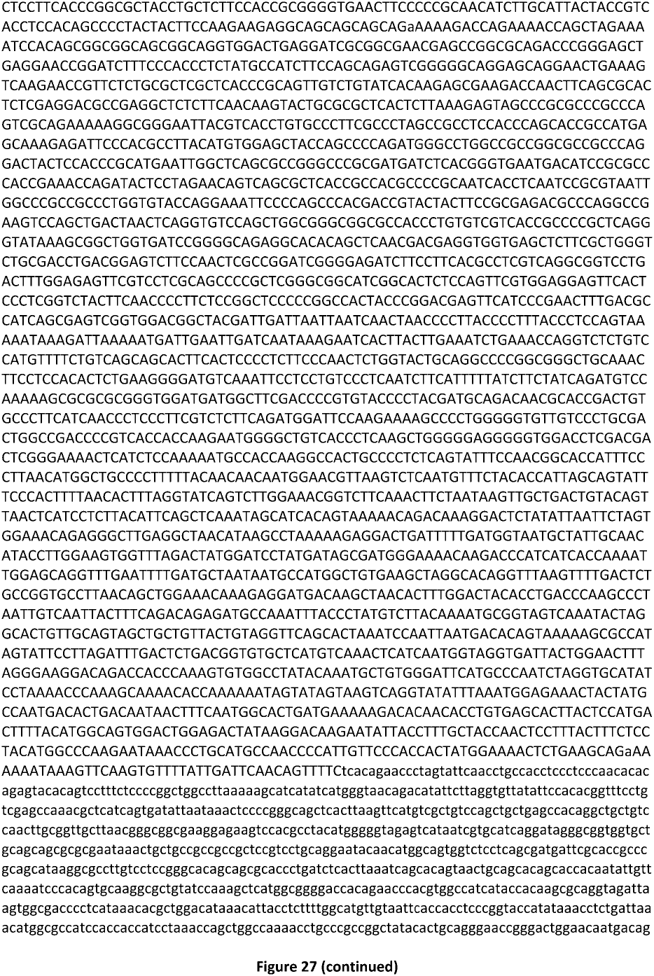

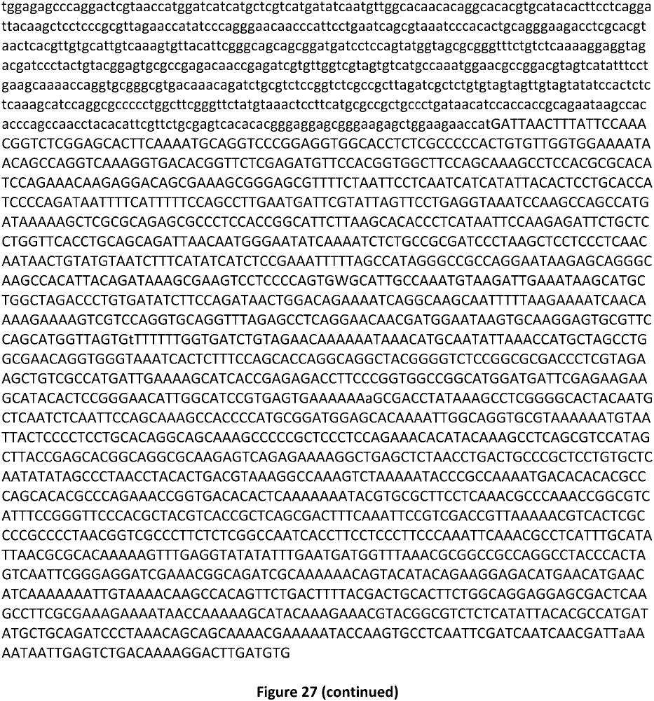

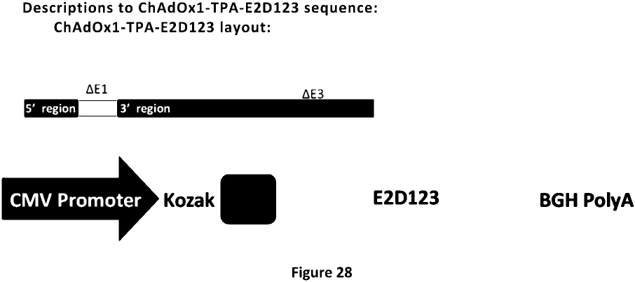

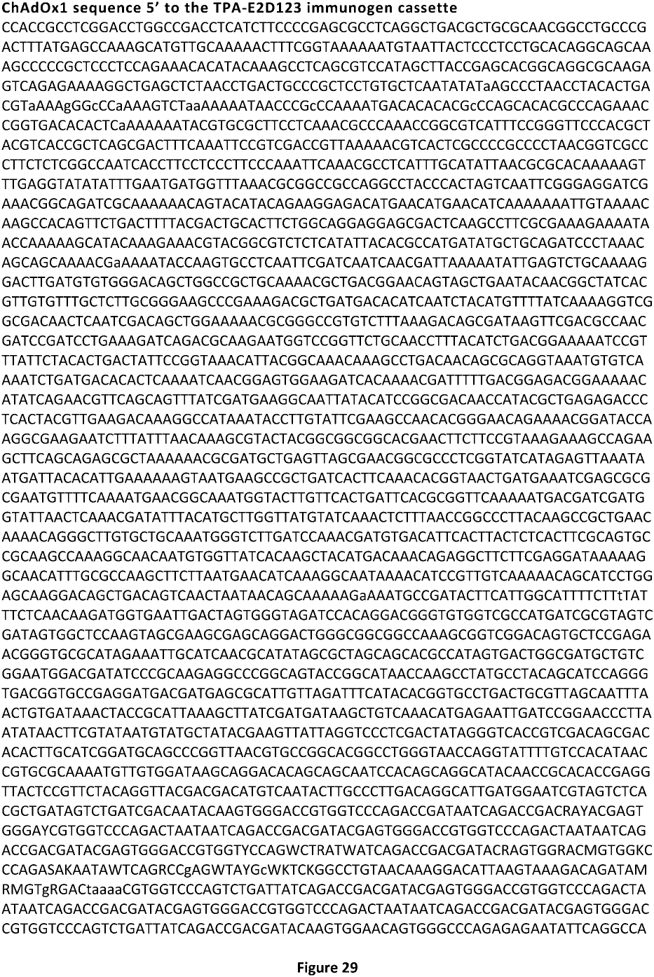

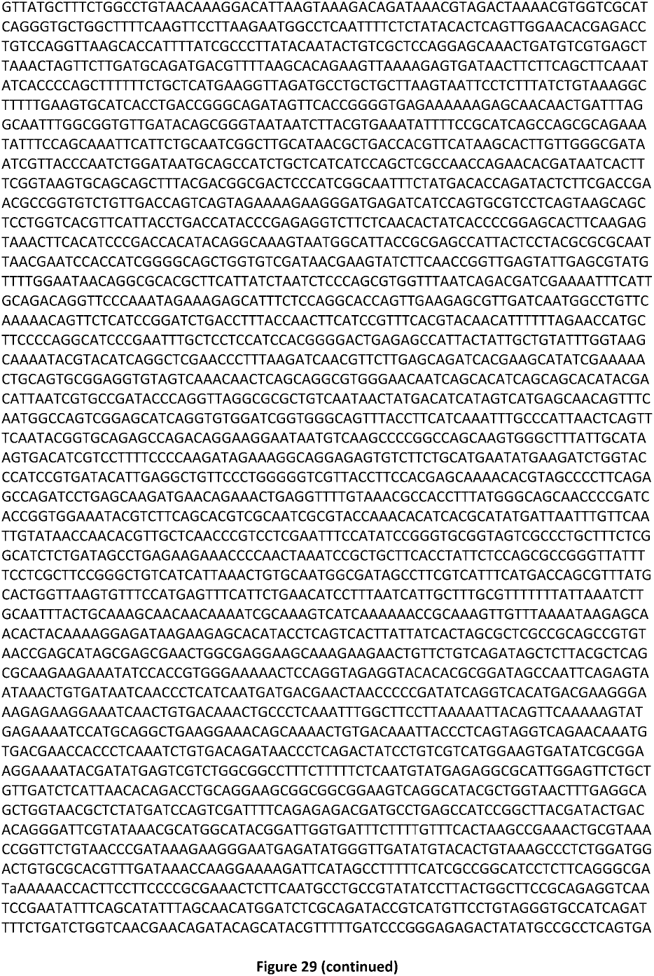

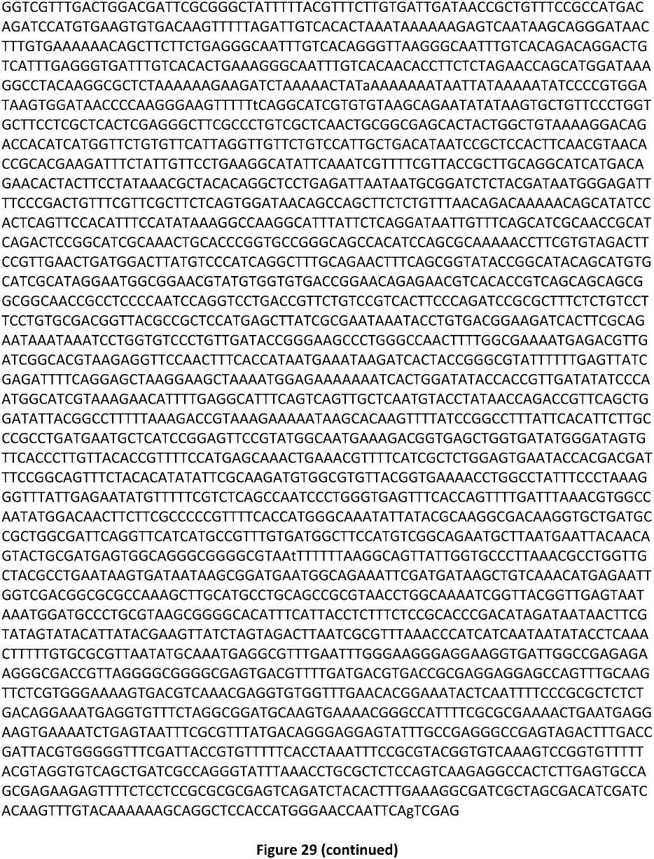

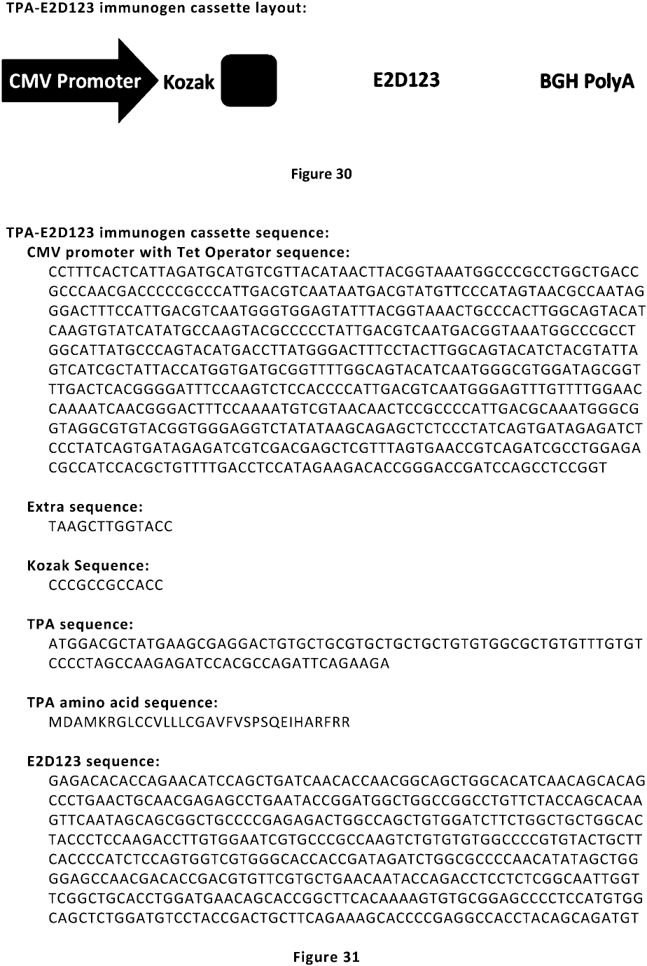

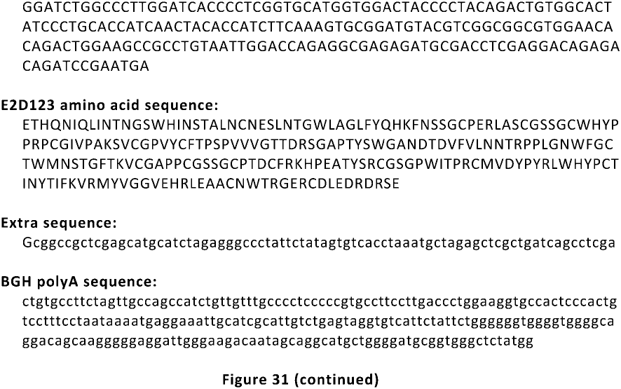

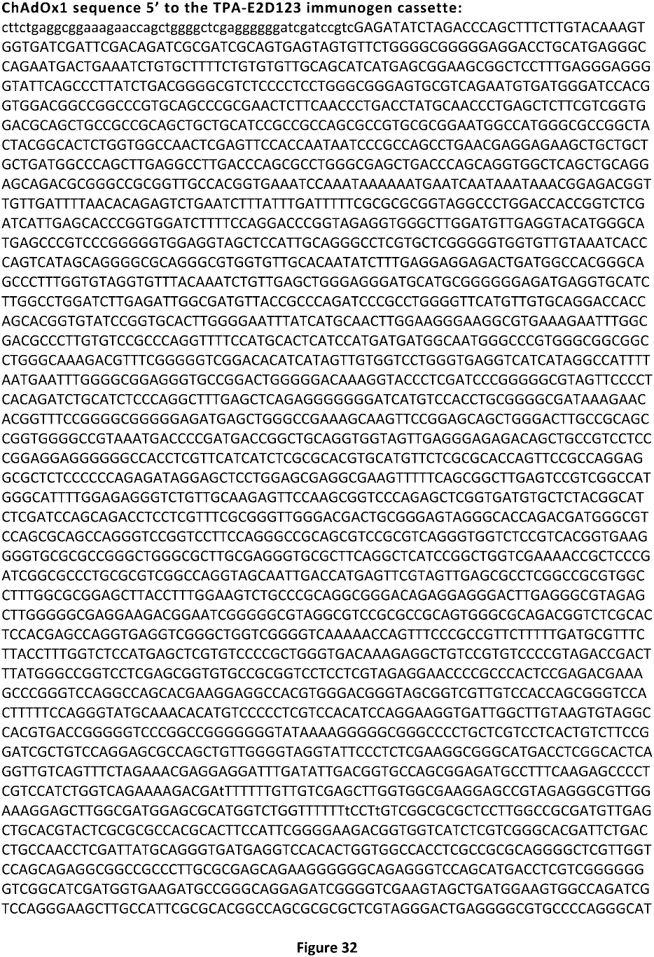

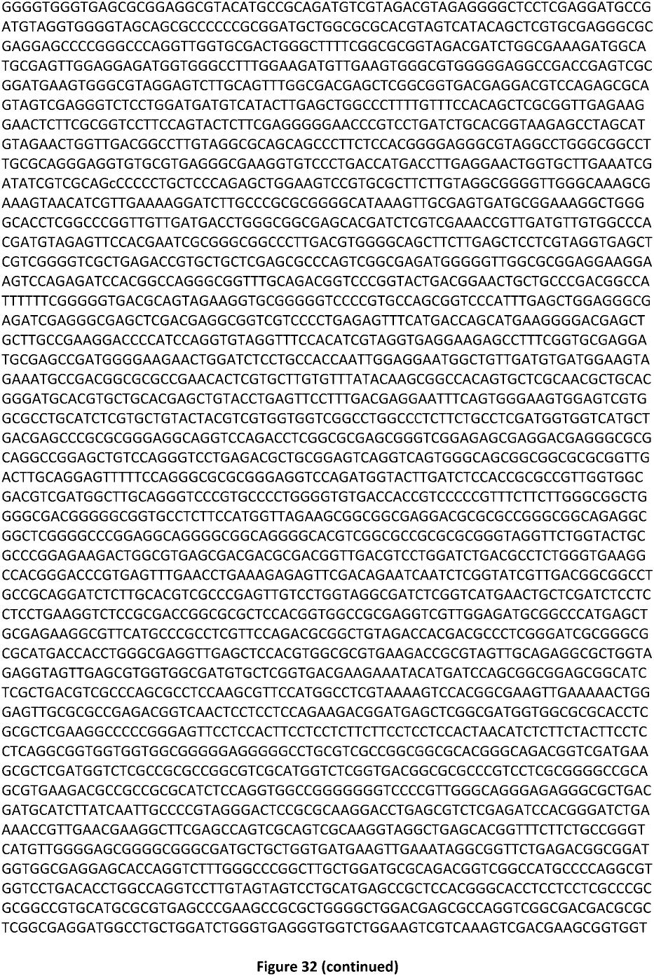

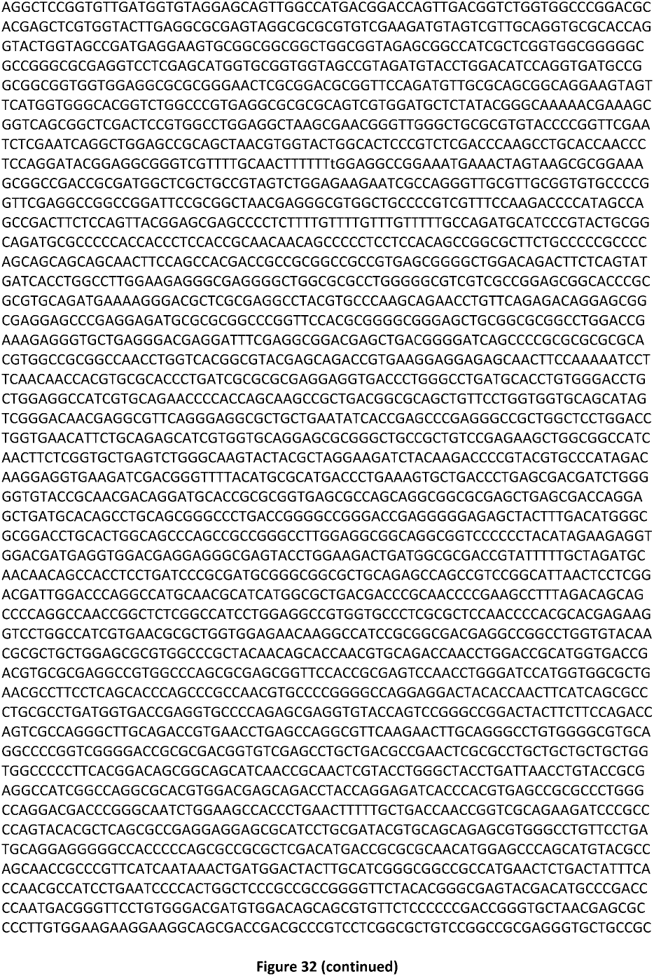

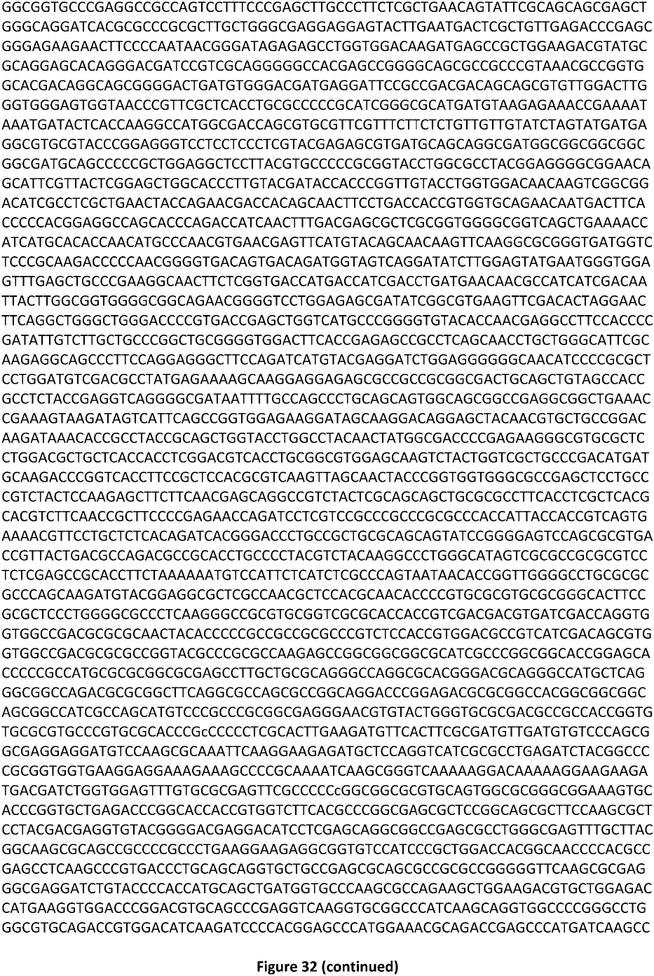

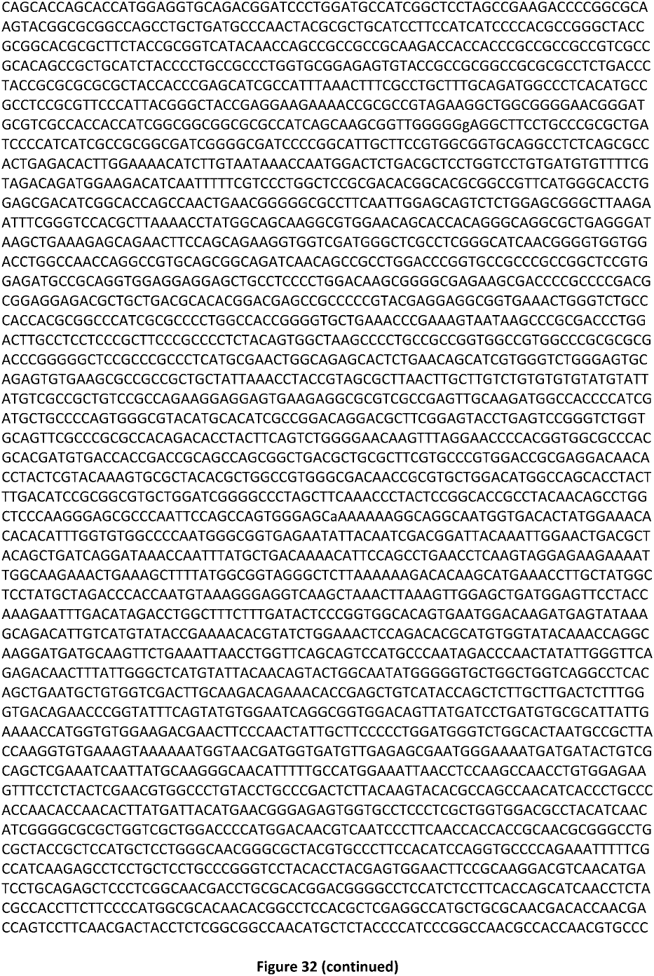

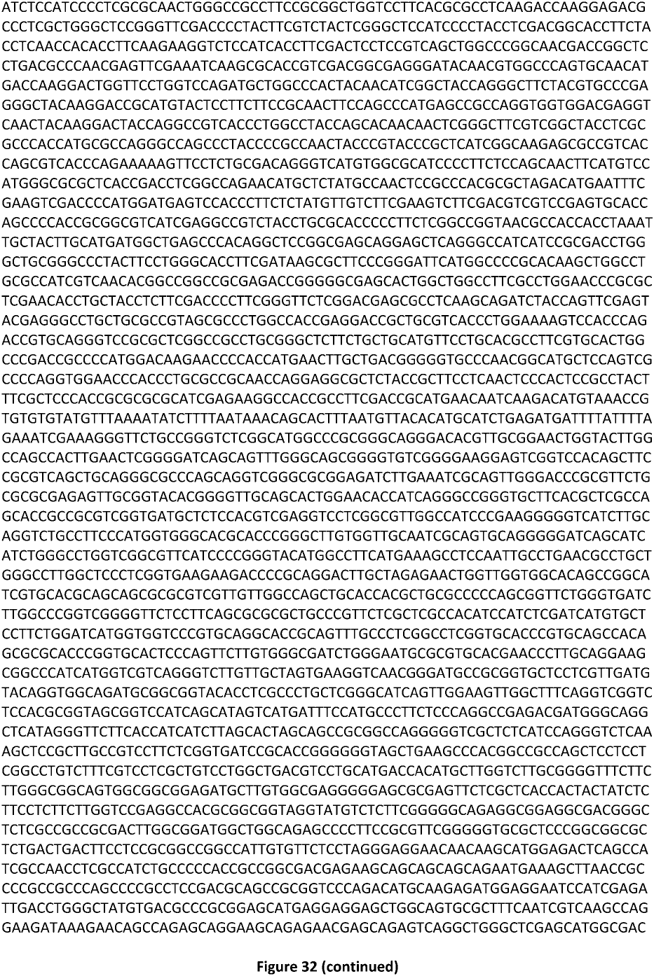

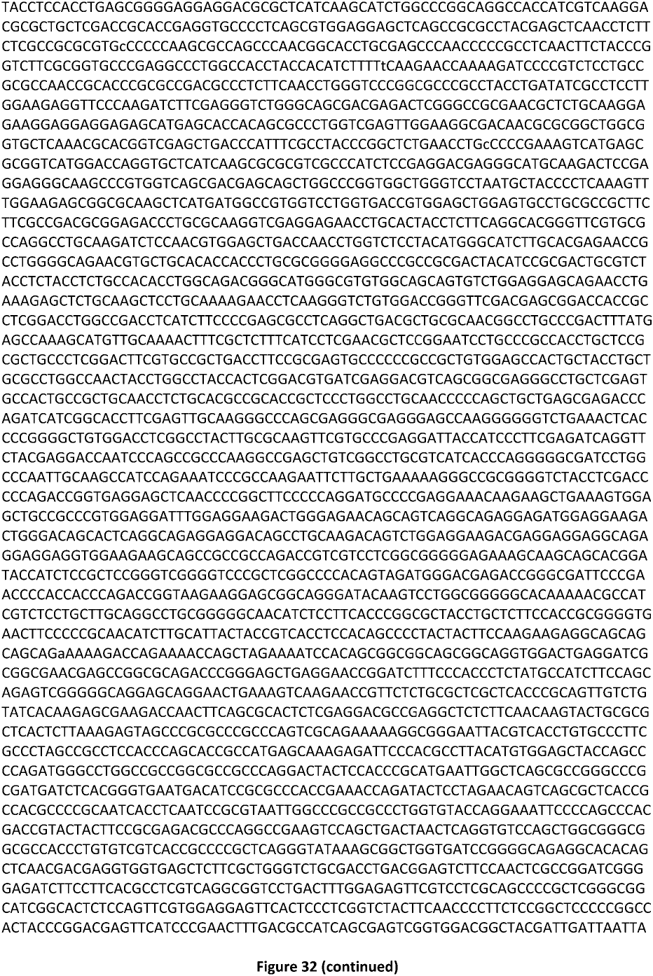

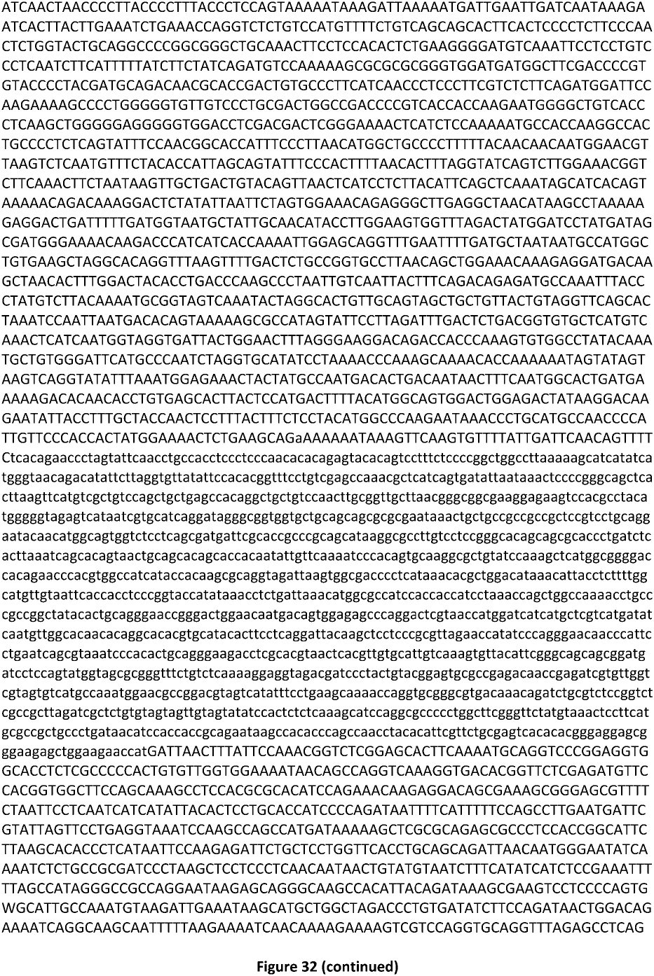

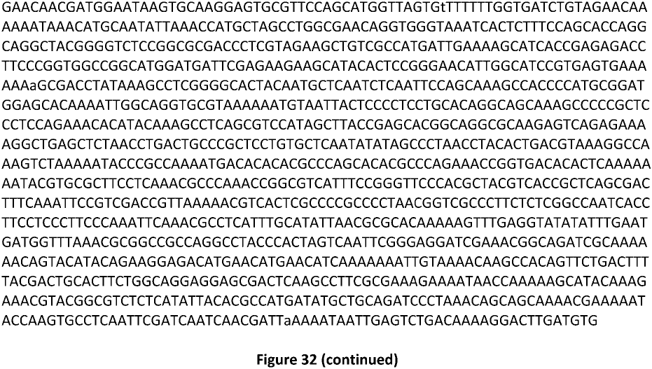

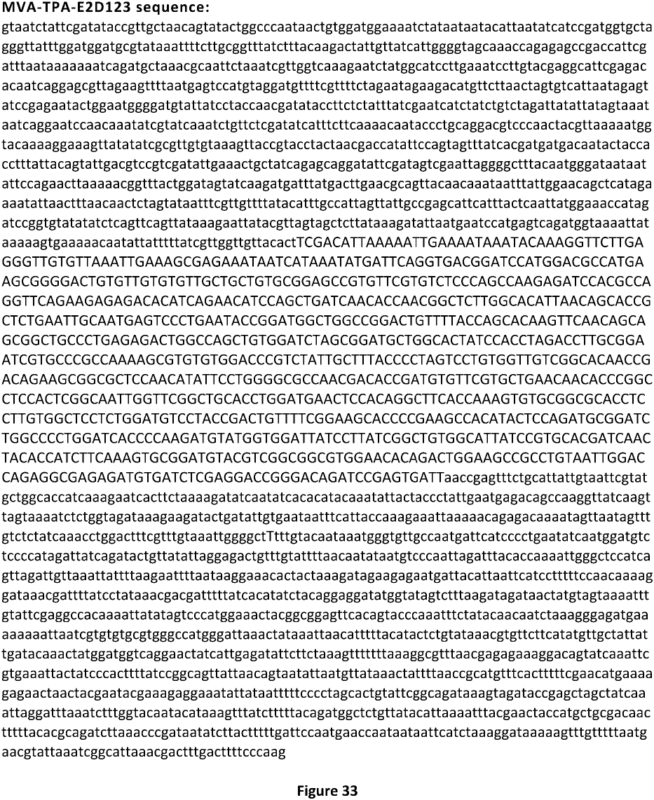

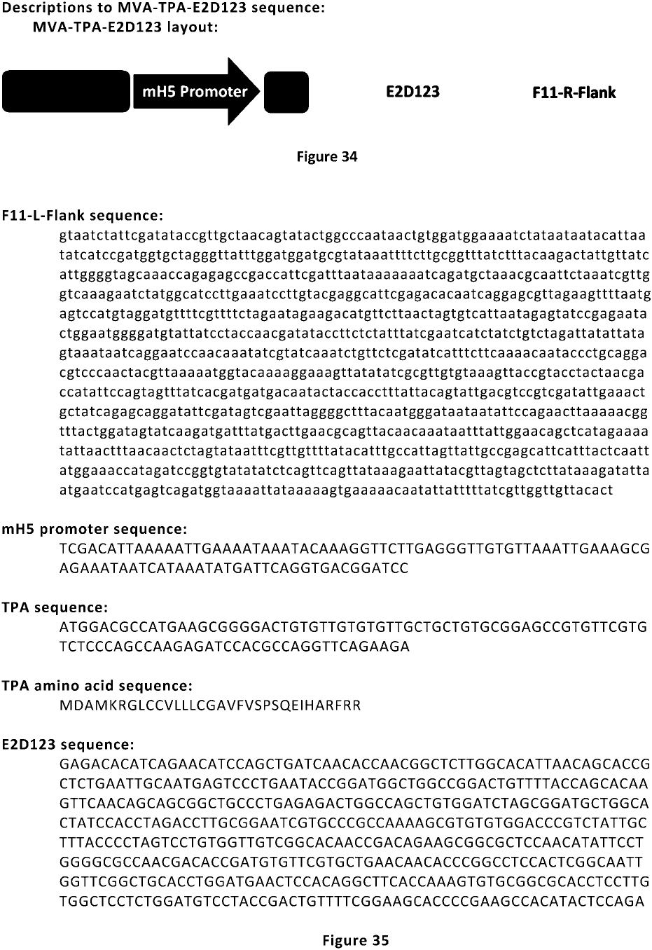

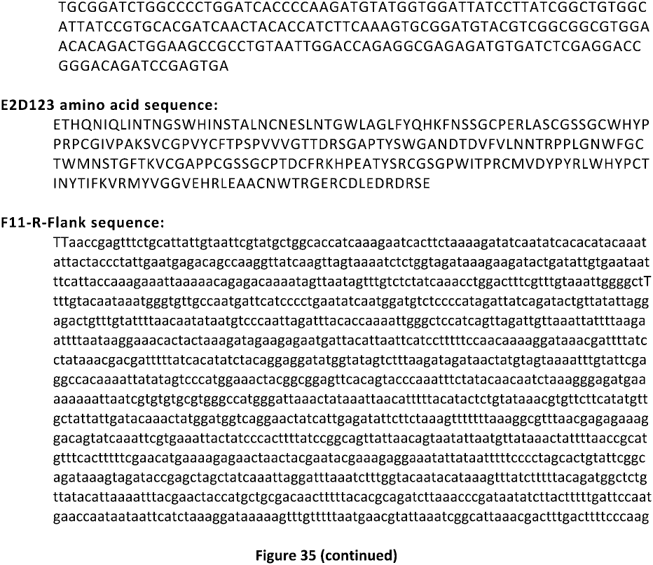

SEQ ID NO:2 D123 DNA sequence of codon optimised for human expression. SEQ ID NO: 3 Sequence information from FIGS. 27 to 35.

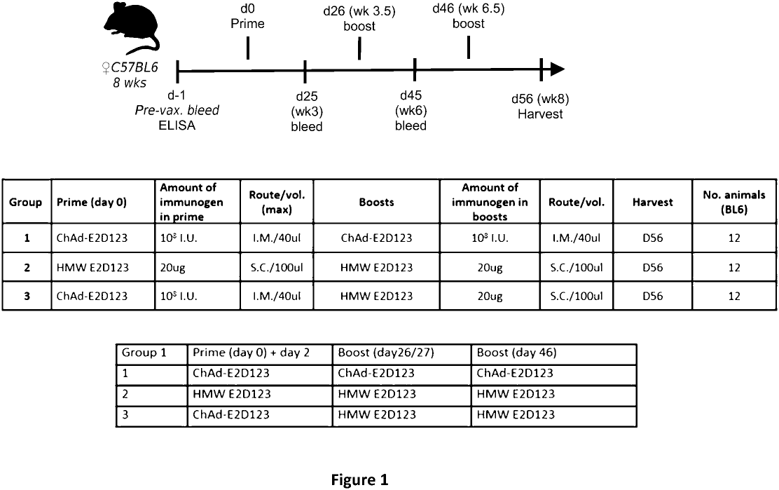

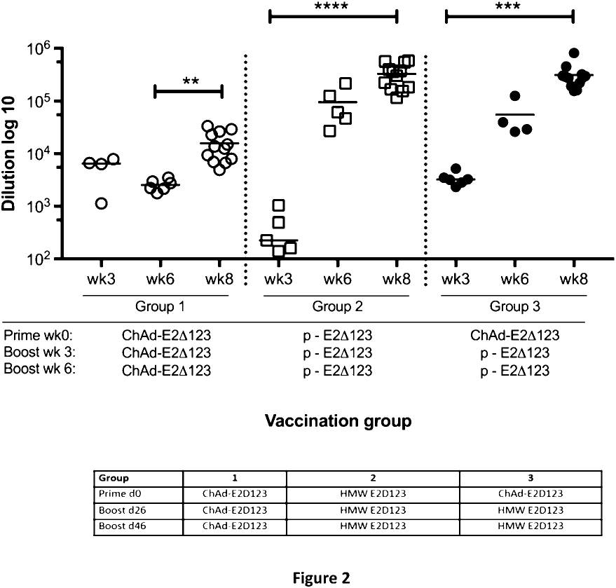

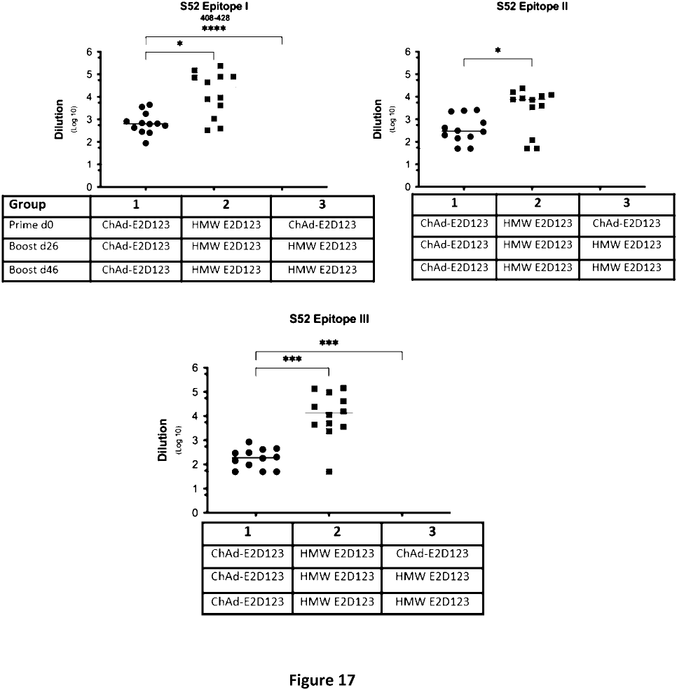

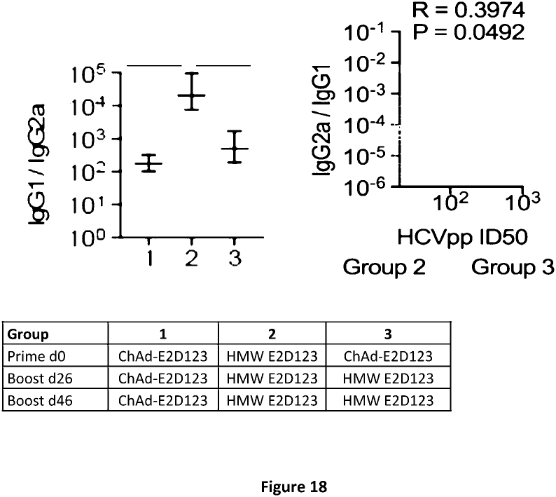

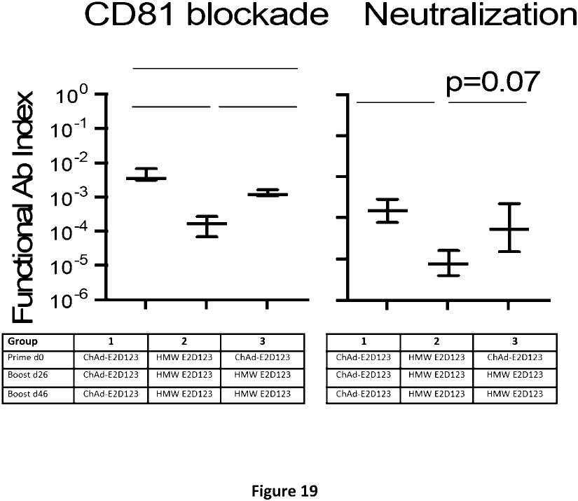

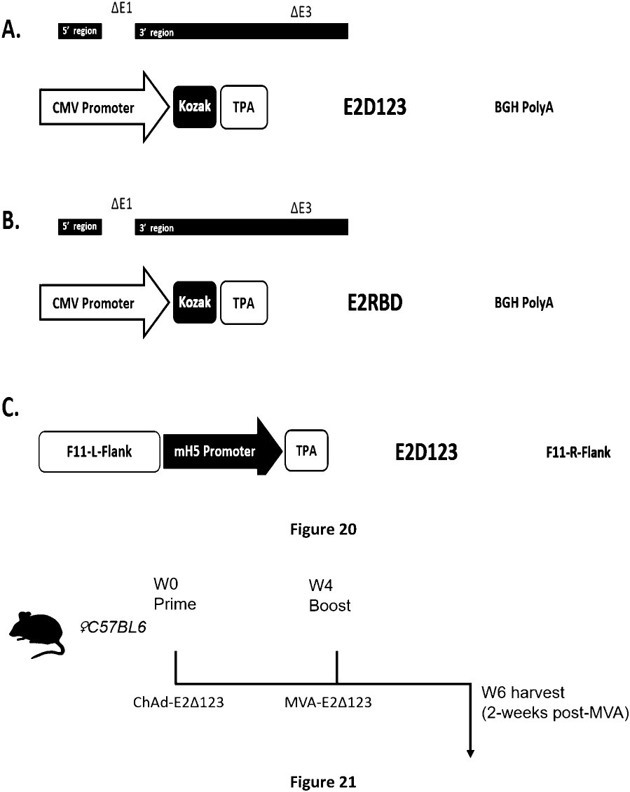

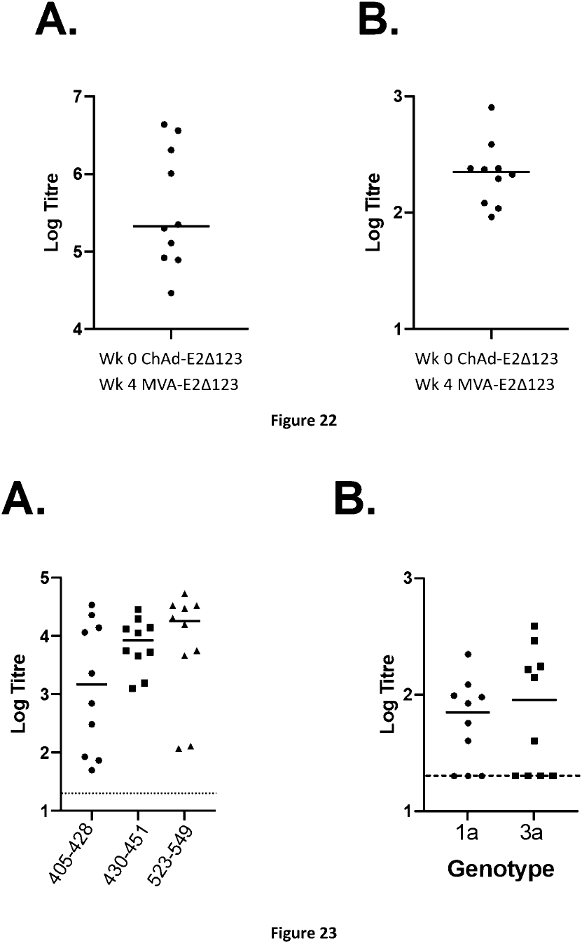

DETAILED DESCRIPTION OF PARTICULAR EMBODIMENTS. It has been identified in accordance with the present invention that a variable region deleted HCV E2 antigen able to elicit a functional B cell response to heterologous HCV genotypes can be administered in the form of a nucleic acid encoding the HCV antigen. Surprisingly, the nucleic acid molecule was able to engender a functional B cell response in a recipient to multiple HCV genotypes. In an illustrative embodiment, the nucleic acid encoding a variable region deleted HCV E2 RBD was administered in a viral vector. It has been demonstrated in mouse vaccination studies that DNA encoding variable region deleted HCV E2 RBD generated the lowest levels of antibody responses compared to administration of protein alone or a combination of protein and DNA, but these antibody responses displayed the highest levels of functional antibodies able to prevent infection such as by preventing E2 binding to its cellular receptor CD81. The DNA vaccines were also greater stimulators of IgG1 and IgG2b, potent activators of complement that assists in viral clearance. Supporting data is presented in Example 1 and FIGS. 1 to 16. Further supporting data are presented in Examples 2 and 3 and in FIGS. 17 to 26 described therein. The specification provides more evidence of the generation of cross reactive antibody specificities in animals vaccinated with ChAdD123 then two protein boosts (FIG. 17), as well as higher levels of functional antibody in the animals that received either three vaccinations with ChAdD123 or a prime with ChAdD123 followed by two protein boosts with HMW E2D123 compared to protein alone (FIG. 19). Evidence of class switching is indicative of a maturation of the B cell response. The ChAd-D123 appears to induce a different class switch to protein vaccination alone and correlates with neutralization (FIG. 18). Example 3 describes evidence that D123 can also be delivered in MVA viral vector (FIGS. 20-26). The experiment was performed by priming mice with ChAdD123 and then boosting with MVA-D123. Animals produced antibodies to E2 (FIG. 22A) which inhibited the interaction between E2 and CD81 22b and were directed to all three major neutralization epitopes (FIG. 23A). Antibodies were neutralizing towards both homologous virus (23B LHS) and heterologous G3a virus (23B RHS).

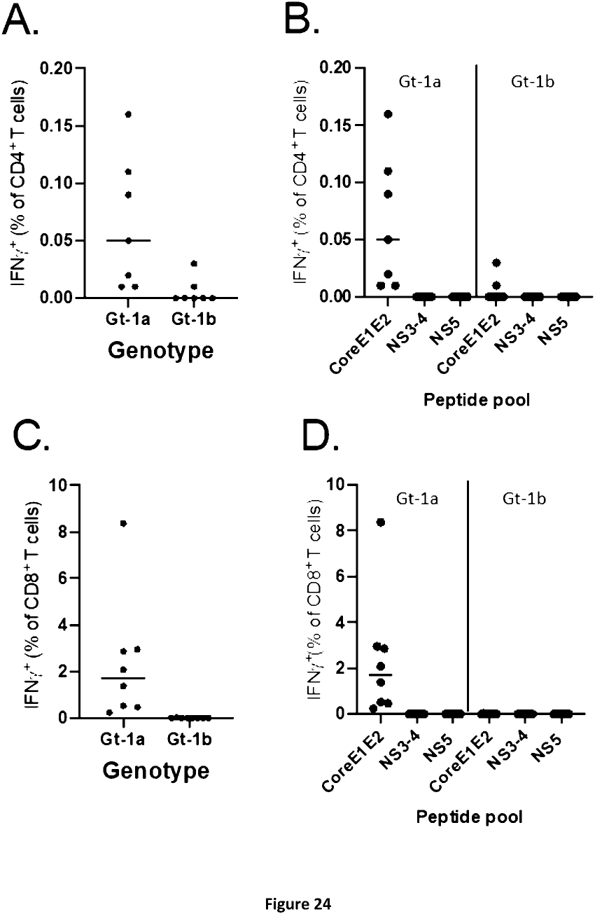

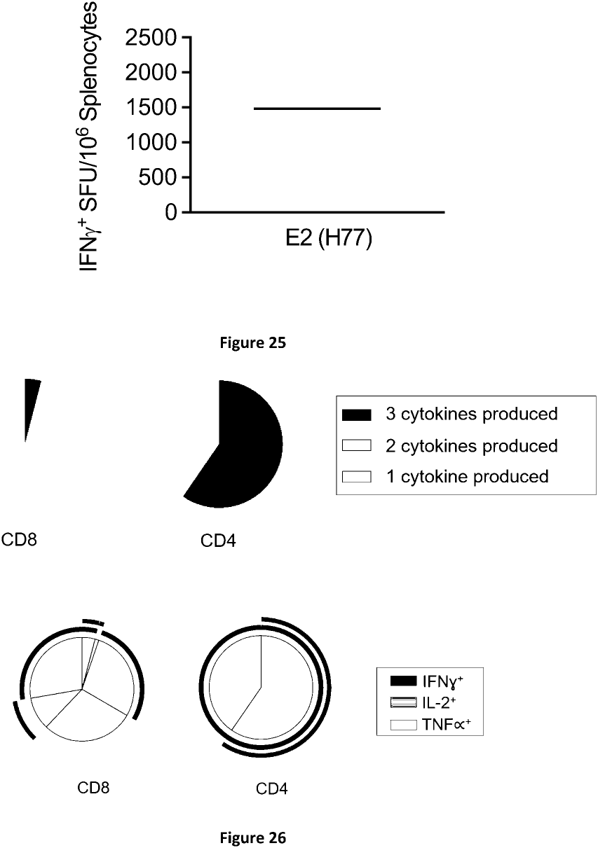

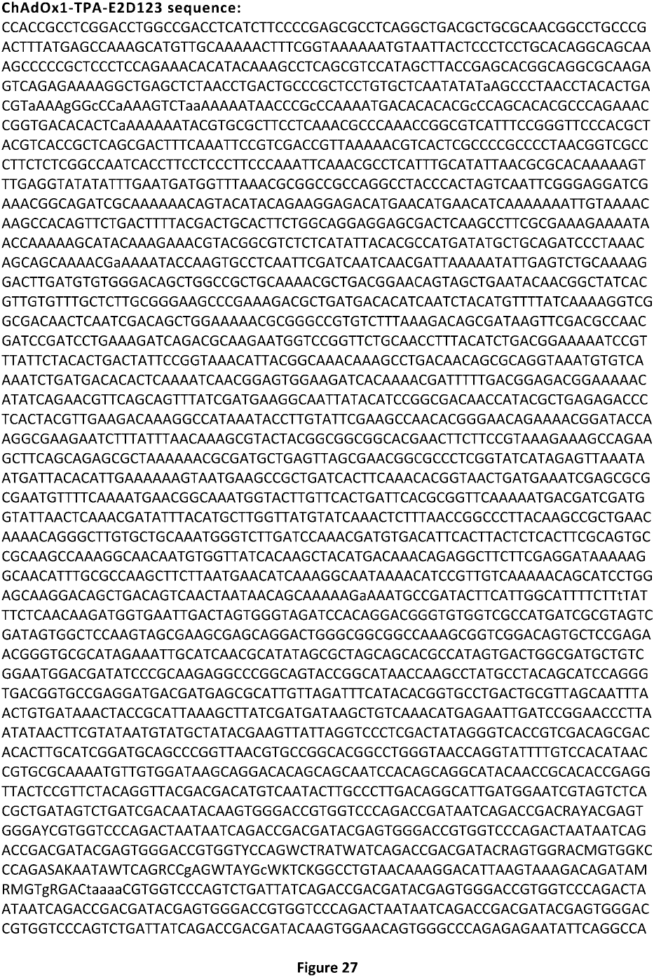

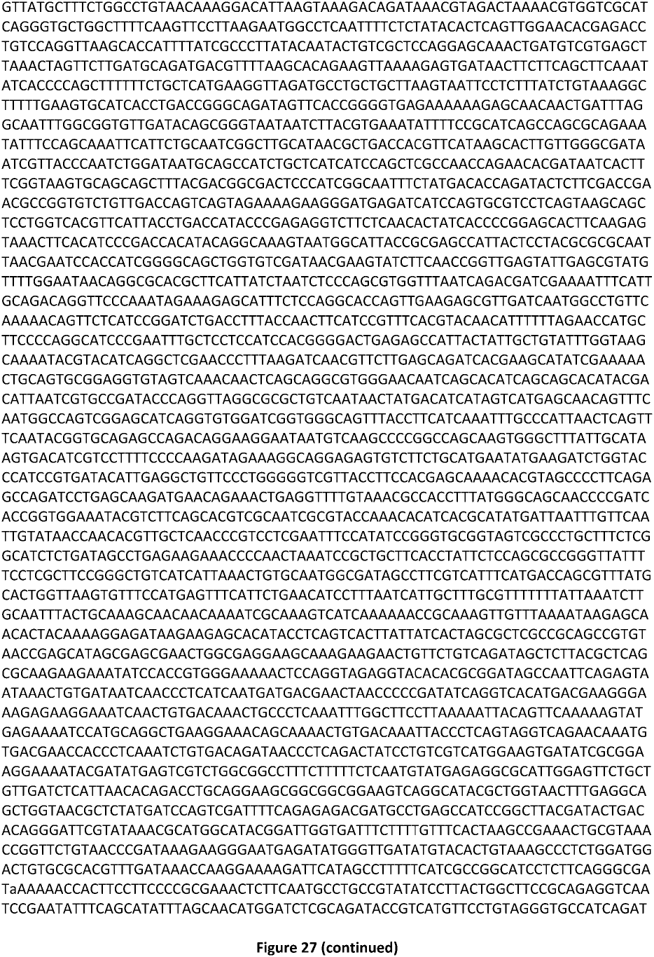

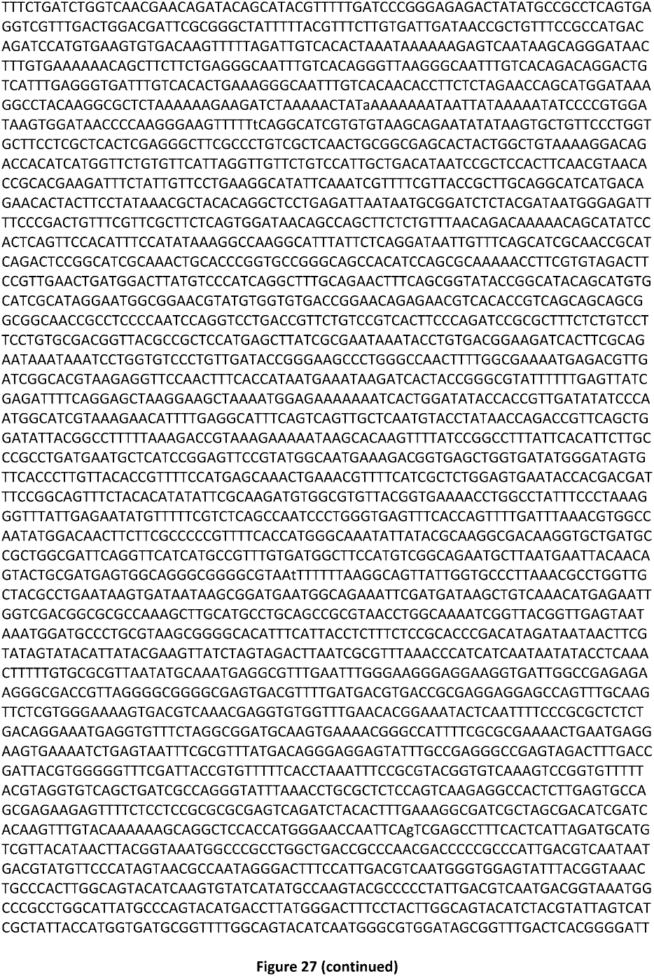

Still furthermore, CD4+ T cell responses were generated towards G1a peptides and some were cross reactive to G3a peptides (FIG. 24A). CD4+ T cell responses were directed towards CoreE1E2 region (FIG. 24B) and some were cross reactive to G3a (FIG. 24B). CD8+ T cell responses were generated towards G1a peptides (FIG. 24A). CD8+ T cell responses were directed towards CoreE1E2 region (FIG. 24B). All T cells were directed towards E2 peptides (FIG. 25). Polyfunctional CD4+ T cell responses were generated making 3 cytokines and CD8+ T cell responses generated predominantly TNFalpha (FIG. 26).

Accordingly, disclosed herein are compositions and methods for vaccinating subjects against current or future HCV infection based on the administration of a nucleic acid based vaccine.

In one aspect, this disclosure provides a method of preventing or reducing HCV infection in a subject comprising administering to the subject a nucleic acid molecule encoding a variable domain deleted HCV E2 proteinaceous molecule.

In one aspect, the disclosure provides a composition comprising a nucleic acid molecule encoding a variable domain deleted E2 polypeptide of HCV for use in therapy or for in the treatment or prevention of HCV infection or for inducing an immune response, wherein the nucleic acid is administered to a subject and wherein the variable domain deleted E2 protein is produced and generates an immune response including a functional B-cell response to HCV in the subject.

Accordingly, in one embodiment the composition is for use as a prophylactic or therapeutic vaccine that engenders an effective functional B-cell response able to reduce or prevent viral propagation in the host and therefore between hosts.

Accordingly, in one embodiment the composition is for use as a vaccine to prevent viral propagation in the host and therefore between hosts.

“Hepatitis C virus” or “HCV” is an small enveloped positive sense, single-stranded RNA virus belonging to the genus hepacivirus of the flaviviridae family. As used herein the term refers to HCV of any genotype, for example, but not limited to strains of HCV genotype 1 (G1), HCV genotype 2 (G2), HCV genotype 3 (G3), HCV genotype 4 (G4), HCV genotype 5 (G5), HCV genotype 6 (G6), HCV genotype 7 (G7), HCV genotype 8 (G8) and can include any subtype or quasispecies thereof e.g. subtype a, b, c, d, e, etc. HCV encodes two glycoproteins E1 and E2 which are required for viral entry into host cells. The terms “HCV E2 glycoprotein”, “E2 glycoprotein”, “E2 dimers”, “E2 trimers” or “E2”, “E2 monomer, “HCV E2” and the like includes an E2 glycoprotein from any genotype or isolate of HCV. The term further includes non-naturally occurring variants including portions of the full length E2 glycoprotein including those that, for example, mediate receptor binding, or mediate neutralizing antibody binding by one or more antibodies that recognize conformational and/or other epitopes, or those portions that mediate E1E2 dimer formation.

The HCV E2 glycoprotein contains ~ 11 largely conserved N-linked glycosylation sites and 18 conserved cysteines that form labile disulfides. The receptor binding domain (RBD) folds independently of other E1E2 sequences and contains the majority of NAb binding sites. The RBD is spanned by amino acid residues 384-661 (G1). The RBD may extend beyond residue 661 to the C-terminal boundary of the ectodomain of E2. Heterologous expression of the E2 RBD results in the secretion of a soluble protein that retains the ability to bind CD81. Located within the RBD are three highly variable regions or HVRs, HVR1 (amino acids 384-410), HVR2 (amino acids 460-481) and the igVR (intergenotypic variable region, 570-580) (McCaffrey etal. J. Virol. 81:9584-9590, 2007). The variable regions perform a major role in immune evasion as they act as immune decoys shielding underlying conserved residues. The “immunoglobulin neutralizing face” or “neutralizing face” refer to a face of E2 that generates immunoglobulins that inhibit the binding of E2 to CD81. The three variable regions can be simultaneously deleted from the RBD without disrupting its native fold indicating that they are outside a conserved core domain. The RBD is in nature linked through a conserved C-terminal stem to the transmembrane domain. X-ray crystallography and electron microscopy revealed that the RBD core adopts a compact immunoglobulin-like fold. The binding site for CD81, the major cellular receptor for all HCV strains, comprises four highly conserved surface exposed segments that overlap with bNAb epitopes (Drummer et al, J. Virol. 76:11143-11147, 2002). The crystal structure confirms that HVR2 and the igVR form disulfide bonded surface exposed loops as proposed previously.

HCV E2 is highly immunogenic, however, few of the antibodies produced against HCV E2 are effective in preventing infection. Antibodies directed towards the N-terminal HVR1 are immunodominant, but are isolate-specific with limited ability to neutralize heterologous strains and genotypes of HCV. As a result, antibodies to HVR1 drive immune escape and HVR1 has been proposed to be an immune decoy. Neutralizing antibodies whose epitopes overlap with sequences involved in CD81 binding are generally broadly neutralizing due to the high degree of sequence conservation in this region but usually only emerge during the chronic phase of infection. Human NAbs that block CD81 binding recognise epitopes located within four immunogenic domains. HVR1 specific antibodies can shield the CD81 binding site and conserved NAb epitopes therein, competing for binding activity and reducing the effectiveness of NAbs to Domain E. High titres of non-NAbs are generated in both natural and vaccine-generated immune responses and map to Domain A, located on the opposite face of E2 from the CD81 binding site. These non-NAbs are immunodominant, fail to prevent E2-CD81 binding, are cross-reactive to multiple genotypes and interfere with the activity of NAbs. These studies revealed that an effective protective antibody response to HCV requires the generation of select antibody specificities that act synergistically to prevent infection.

As used herein “CD81” refers to cluster of differentiation 81 is a transmembrane protein of the tetraspanin superfamily and is the major host cell receptor for different HCV strains.

As used herein “Δ123 E2 ” or Delta123E2” refers to an E2 polypeptide where HVR1, HVR2 and igVR/VR3 have been removed or deleted and optionally replaced with linkers as generally described in McCaffrey et al. 2012. In one embodiment, the invention has been illustrated with nucleic acids encoding E2Delta123 wherein each of the variable domain encoding regions have been removed. While this embodiment likely to be the most efficacious, however, it will be appreciated that some partial regions or portions of polynucleotides encoding variable regions could be retained and still elicit enhanced neutralising and diminished non-neutralizing antibody responses relative to an intact control E2 polypeptide comprising variable regions, and these embodiments are encompassed. Thus, for example, it is envisaged in one embodiment that between 1% and 50% of the total variable region polynucleotide sequences could be retained and still fall within the scope of the invention. In one embodiment, less than 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less than 1% E2 variable domain polynucleotides are retained. In one embodiment less than 20%, 19%, 18%, 17%, 16%, 15%, 14%, 13%, 12%, 11%, 10%, 9%, 8%, 7%, 6%, 5%, 4%, 3%, 2%, 1% or less than 1% E2 variable domain polynucleotides are retained. In one embodiment less than 10%, or less than 5%, 4%, 3%, 2%, 1% or less than 1% E2 variable domain polynucleotides are retained. The Δ123 polypeptide retains CD81 binding capacity and can exist in monomeric, dimeric, trimeric and various oligomeric forms. In one embodiment, the HMW forms may be HMW1 or HMW2. As used herein “HMW1” refers to an oligomeric form of Δ123 with a molecular weight of ~2402 kDa. As used herein “HMW2” refers to an oligomeric form of Δ123 with a molecular weight of ~239.3 kDa.

One illustrative form of HCV E2 glycoprotein is a receptor binding portion of E2 glycoprotein comprising amino acids 384-661 of genotype H77 1a (E2₆₆₁) or a corresponding portion from another HCV genotype.

As used herein “genotype specific antibodies” refers to antibodies that bind to a single HCV genotype or two very similar genotypes. A person skilled in the art would understand that the HCV genotype can be any HCV genotype. In one embodiment, the HCV genotype is selected from one or more of: G1, G2, G3,G4, G5, G6, and G7.

In one embodiment, the E2 protein is E2₆₆₁Δ123 (AHVR123, also referred to herein as Δ123, D123, AHVR1+2+3, D123 E2₆₆i-his, DI23 E2₆₆₁, Δ123 E2₆₆₁and Δ123 E2₆₆₁-his). In some embodiments, the E2₆₆₁Δ123 E2 comprises amino acids 384-661 of HCV H77c where the variable regions 1 and 2 and igVR (3) are replaced with short linker motifs (AHVR123). Other embodiments of the invention include a HCV E2 protein having any combination of HVR1, HVR2 or igVR deleted or replaced by a short linker. These can be abbreviated as Δ1 ; Δ1,2 (Δ12); Δ2; Δ2,3 (Δ23); Δ3; and Δ1,3 (Δ13).

The term “isolated” and “purified” means material that is substantially or essentially free from components, that normally accompany it in its native state. For example, an “isolated nucleic acid molecule” refers to a nucleic acid or polynucleotide, isolated from the sequences which flank it in a naturally-occurring state, e.g., a DNA fragment which has been removed from the sequences that are normally adjacent to the fragment. In particular, an isolated HCV E2 includes in vitro isolation and/or purification of a protein from its natural cellular environment, and from association with other components of the cell. Without limitation, an isolated nucleic acid, polynucleotide, peptide, or polypeptide can refer to a native sequence that is isolated by purification or to a sequence that is produced by recombinant or synthetic means.

As used herein “broadly neutralizing antibodies” refers to antibodies that provide cross protection against multiple genotypes or subtypes of an immunogen/HCV. In an embodiment, the broadly neutralizing antibodies recognize more than twogenotype and/or subtype of HCV. In an embodiment, the broadly neutralizing antibodies recognize at least 3, or at least 4, or at least 5, or at least 6, or at least 7 or at least 8 HCV genotypes. In an embodiment, the HCV genotypes is selected from: G1, G2, G3, G4, G5, G6, G7. In an embodiment, the broadly neutralizing antibodies bind 3 HCV genotypes. In an embodiment, the broadly neutralizing antibodies bind 4 HCV genotypes. In an embodiment, the broadly neutralizing antibodies bind 5 HCV genotypes. In an embodiment, the broadly neutralizing antibodies bind 6 HCV genotypes. In an embodiment, the broadly neutralizing antibodies bind 7 HCV genotypes. In an embodiment, the broadly neutralizing antibodies bind other HCV genotypes. A person skilled in the art would appreciate that broadly neutralizing antibodies may take weeks or months to develop in a subject.

A nucleic acid molecule as described herein may in any form such as DNA or RNA, including in vitro transcribed RNA or synthetic RNA, mRNA or PNA or a mixture thereof. Nucleic acids include genomic DNA, cDNA, mRNA, recombinantly produced and chemically synthesized molecules and modified forms thereof. A nucleic acid molecule may be single stranded or double stranded and linear or closed covalently to form a circle. The RNA may be modified by stabilizing sequences, capping, and polyadenylation. RNA or DNA and may be delivered as plasmids to express antigen and induce immune responses. RNA-based approaches are generally preferred and these can include amplifying or non-self amplifying constructs.

In some embodiments the polynucleotide to be administered by transient in vivo transfection is a chemically modified RNA in which a proportion (e.g., 10%, 30%, 50%, or 100%) of at least one type of nucleotide, e.g., cytosine, is chemically modified to increase its stability in vivo. For example, in some cases modified cystosines are 5-methylcytosines. Such polynucleotides are particularly useful for delivery/transfection to cells in vivo, especially when combined with a transfection/delivery agent. In some cases, a chemically modified RNA is a chemically modified RNA in which a majority of (e.g., all) cystosines are 5-methylcytosines, and where a majority (e.g., all) of uracils are pseudouracils. The synthesis and use of such modified RNAs are described in, e.g., WO 2011/130624. Methods for in vivo transfection of DNA and RNA polynucleotides are known in the art as summarised in, e.g., Liu et al (2015) and Youn et al (2015).

The term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

Accordingly, in one embodiment, the G/C content of the coding region of the nucleic acid coding region is modified, particularly increased, compared to the G/C content of the coding region of its particular wild type coding sequence, i.e. the unmodified mRNA. The encoded amino acid sequence of the mRNA is preferably not modified compared to the coded amino acid sequence of the particular wild type mRNA.

An optimised mRNA based composition could comprise a 5′ and 3′ non translated region (5′-UTR, 3′-UTR) that optimise translation efficiency and intracellular stability as known in the art and an open reading frame encoding the variable region deleted HCV E2. In one embodiment, removal of uncapped 5′-triphosphates can be achieved by treating RNA with a phosphatase. RNA may have modified ribonucleotides in order to increase its stability and/or decrease cytotoxicity. For example, in one embodiment, in the RNA, 5-methylcytidine is substituted partially or completely, for cytidine. Alternatively or additionally, pseudouridine is substituted partially or completely, preferably completely, for uridine. These modification may also reduce indiscriminate immune inactivation which may hinder translation of the RNA. In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap. The term “5′-cap” includes a 5′-cap analog that resembles the RNA cap structure and is modified to possess the ability to stabilize RNA and/or enhance translation of RNA. Providing an RNA with a 5′-cap or 5′-cap analog may be achieved by in vitro transcription of a DNA template in the presence of said 5′-cap or 5′-cap analog, wherein said 5′-cap is co- transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

A further modification of RNA may be an extension or truncation of the naturally occurring UTR such as the X-region tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alphal-globin, beta-globin. RNA having an unmasked poly-A sequence is translated more efficiently than RNA having a masked poly-A sequence.

The term “poly(A) tail” or “poly-A sequence” relates to a sequence of adenyl (A) residues which may be located on the 3′-end of a RNA molecule and “unmasked poly-A sequence” means that the poly-A sequence at the 3′ end of an RNA molecule ends with an A of the poly-A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA it may be modified so as to be present in conjunction with a heterologous poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase stability and/or expression of the RNA used according to the invention, the poly-A sequence can be unmasked.

In addition, incorporation of a 3′-non translated region (UTR) into the 3′-non translated region of an RNA molecule can result in an enhancement in translation efficiency. A synergistic effect may be achieved by incorporating two or more of such 3′-non translated regions. The 3′-non translated regions may be autologous or heterologous to the RNA into which they are introduced. In one particular embodiment the 3′-non translated region is derived from the human β-globin gene.

A combination of the above described modifications, i.e. optionally incorporation of a poly-A sequence, unmasking of the poly-A sequence and incorporation of one or more 3 ′-non translated regions, has a synergistic influence on the stability of RNA and increase in translation efficiency.

In order to increase expression of the RNA it may be modified within the coding region so as to increase the GC-content to increase mRNA stability and to perform a codon optimization and, thus, enhance translation in cells. Modified mRNA may be synthesised enzymatically and packaged into nanoparticles such as lipid nanoparticles and administered, for example intramuscularly. Self-replicating RNA or protamine complexed RNA approaches have also been shown to generate immune responses against viral infections.

The nucleic acid molecule can be entrapped in microcapsules prepared, for example, by coacervation techniques or by interfacial polymerization, in colloidal drug delivery systems (e.g., liposomes, microspheres, microemulsions, nanoparticles and nanocapsules), or in macroemulsions. Such techniques are known in the art and disclosed in Remington, the Science and Practice of Pharmacy, 20th Edition, Remington, J., ed. (2000).

Various approaches for systemic administration of nucleic acids as nanoparticles or colloidal systems are known. In non-viral approaches, cationic liposomes are used to induce DNA/RNA condensation and to facilitate cellular uptake. The cationic liposomes usually consist of a cationic lipid, like DOTAP, and one or more helper lipids, like DOPE. So-called ‘lipoplexes’ can be formed from the cationic (positively charged) liposomes and the anionic (negatively charged) nucleic acid. In the simplest case, the lipoplexes form spontaneously by mixing the nucleic acid with the liposomes with a certain mixing protocol, however various other protocols may be applied. In one embodiment, nanoparticulate RNA formulations such as RNA lipoplexes, are produced with defined particle size wherein the net charge of the particles is close to zero or negative. For example, electro-neutral or negatively charged lipoplexes from RNA and liposomes lead to substantial RNA expression in spleen or immune cells after systemic administration as disclosed in WO2013/143683. In one embodiment, the nanoparticles comprise at least one lipid. In one embodiment, the nanoparticles comprise at least one cationic lipid. The cationic lipid can be monocationic or polycationic. Any cationic amphiphilic molecule, eg, a molecule which comprises at least one hydrophilic and lipophilic moiety is a cationic lipid within the meaning of the present invention. In one embodiment, the positive charges are contributed by the at least one cationic lipid and the negative charges are contributed by the RNA. In one embodiment, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In one embodiment, the cationic lipid and/or the helper lipid is a bilayer forming lipid.

In one embodiment, the at least one cationic lipid comprises 1,2-di-0-octadecenyl-3-trimethylammonium propane (DOTMA) or analogs or derivatives thereof and/or 1 ,2-dioleoyl-3-trimethylammonium-propane (DOTAP) or analogs or derivatives thereof. In one embodiment, the at least one helper lipid comprises 1 ,2-di-(9Z-octadecenoyl)-sn-glycero-3-phosphoethanolamine (DOPE) or analogs or derivatives thereof, cholesterol (Choi) or analogs or derivatives thereof and/or 1,2-dioleoyl-sn-glycero-3-phosphocholine (DOPC) or analogs or derivatives thereof.

In one embodiment, the molar ratio of the at least one cationic lipid to the at least one helper lipid is from 10:0 to 3:7, preferably 9: 1 to 3:7, 4: 1 to 1 :2, 4: 1 to 2:3, 7:3 to 1 : 1, or 2: 1 to 1 : 1 , preferably about 1 : 1. In one embodiment, in this ratio, the molar amount of the cationic lipid results from the molar amount of the cationic lipid multiplied by the number of positive charges in the cationic lipid. In the nanoparticles described herein the lipid may form a complex with and/or may encapsulate the RNA. In one embodiment, the nanoparticles comprise a lipoplex or liposome. In one embodiment, the lipid is comprised in a vesicle encapsulating said RNA. The vesicle may be a multilamellar vesicle, an unilamellar vesicle, or a mixture thereof. The vesicle may be a liposome.

The antigen encoding sequence may be inserted into any suitable vector. The purpose of the vector is at least in one embodiment to transmit the encoding nucleic acid to the host environment to facilitate protein expression and presentation to the host immune response. Vectors may be replicating or non-replicating.

The term “vector” as used herein includes any transmitting moiety into which the antigen encoding sequence at least in inserted, including plasmid vectors, cosmid vectors, phage vectors such as lambda phage, viral -like particles, viral vectors such as such as adenoviruses, adeno-associated viruses (AAV), alphaviruses, flaviviruses, herpes simplex viruses (HSV), measles viruses, CMV, rhabdoviruses, retroviruses, lentiviruses, Newcastle disease virus (NDV), poxviruses, and picornaviruses or baculoviral vectors, or artificial chromosome vectors such as bacterial artificial chromosomes (BAC), yeast artificial chromosomes (YAC), or PI artificial chromosomes (PAC). Vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

Expression vectors generally include transcriptional and translational regulatory nucleic acid operably linked to the nucleic acid molecule encoding the E2 polypeptide. “Operably linked” in this context means that the transcriptional and translational regulatory DNA is positioned relative to the coding sequence of the E2 polypeptide in such a manner that transcription is initiated. Generally, this will mean that the promoter and transcriptional initiation or start sequences are positioned 5′ to the protein coding region. The transcriptional and translational regulatory nucleic acid will generally be appropriate to the cell used to express the exogeneous protein; for example, transcriptional and translational regulatory nucleic acid sequences from mammalian cells, and particularly humans, are preferably used to express the protein in mammals and humans. Numerous types of appropriate expression vectors, and suitable regulatory sequences are known in the art.

The viral vector may comprise Modified Vaccinia Ankara (MVA). The viral vector may comprise MVA when used as a vaccine boost in a prime boost regime . The viral vector may comprise Adeno-associated virus (AAV) or lentivirus. The viral vector may be an attenuated viral vector.

Unless defined otherwise, all technical and scientific terms used herein have the same meanings as commonly understood by one of ordinary skill in the art to which this disclosure belongs. Any materials and methods similar or equivalent to those described herein can be used to practice or test the present disclosure. Practitioners are particularly directed to and Ausubel et al., Current Protocols in Molecular Biology, Supplement 47, John Wiley & Sons, New York, 1999; Colowick and Kaplan, eds., Methods In Enzymology, Academic Press, Inc.; Weir and Blackwell, eds., Handbook of Experimental Immunology, Vols. I-IV, Blackwell Scientific Publications, 1986; Remington, the Science and Practice of Pharmacy, 20th Edition, Remington, J., ed. (2000) for definitions and terms of the art and other methods known to the person skilled in the art.

In another embodiment, the composition further comprises a pharmaceutically or physiologically acceptable carrier or diluent.

Pharmaceutical compositions are conveniently prepared according to conventional pharmaceutical compounding techniques. See, for example, Remington, the Science and Practice of Pharmacy, 20th Edition, Remington, J., ed. (2000). later editions. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral or parenteral.

The vaccine compositions described in this specification can be combined with other vaccines or treatments. Existing anti-HCV drug treatments are highly effective and known in the art. Agents to improve the immune response by, for example, check point inhibitors, or by including other adjuvant -type molecules are contemplated for use in combination with the subject vaccines.

In one embodiment the nucleic acid molecule is administered in combination with an adjuvant.

In one embodiment the nucleic acid molecule is administered without traditionally used vaccine adjuvants.

Immune responses to immunogens can be enhanced if administered as a mixture with one or more adjuvants. Immune adjuvants typically function in one or more of the following ways: (1) immunomodulation (2) enhanced presentation (3) CTL production (4) targeting; and/or (5) depot generation.

Illustrative adjuvants that may or may not be included include: particulate or non-particulate adjuvants, complete Freund’s adjuvant (CFA), aluminum salts, emulsions, ISCOMS, LPS derivatives such as MPL and derivatives thereof such as 3D-MPL also GLA, and AGP, mycobacterial derived proteins such as muramyl di- or tri-peptides, particular saponins from Quillaja saponaria, such as QS21, QS7, and ISCOPREP™ saponin, ISCOMATRIX™ adjuvant, and peptides, such as thymosin alpha 1. In addition to the saponin component, the adjuvant may comprises a sterol such as beta-sitosterol, stigmasterol, ergosterol, ergocalciferol and cholesterol. In some embodiments, the adjuvant is presented in the form of an oil-in-water emulsion, e.g. comprising squalene, alpha-tocopherol and a surfactant (see e.g. W095/17210) or in the form of a liposome. The term “liposome” when used herein refers to uni- or multilamellar lipid structures enclosing an aqueous interior. Liposomes and liposome formulations are well known in the art. Liposomal presentations are e.g. described in WO 96/33739 and WO2007/068907. Lipids which are capable of forming liposomes include all substances having fatty or fat-like properties. Dynamic laser light scattering is a method used to measure the size of liposomes well known to those skilled in the art. An extensive description of adjuvants can be found in Cox and Coulter, “Advances in Adjuvant Technology and Application”, in Animal Parasite Control Utilizing Biotechnology, Chapter 4, Ed. Young, W.K., CRC Press 1992, and in Cox and Coulter, Vaccine 15(3): 248- 256, 1997.

In accordance with these embodiments, the composition is generally administered for a time and under conditions sufficient to elicit an immune response comprising the generation of functional E2-specific antibodies. The compositions of the present invention may be administered as a single dose or application. Alternatively, the compositions may involve repeat doses or applications, for example the compositions may be administered 2, 3, 4, 5, 6, 7, 8, 9, 10 or more times.

An example of a vaccination regimen contemplated by the present application is as follows: Following an initial vaccination, subjects typically receive a boost after a 2 to 4 week interval, for example a 3 week interval, optionally followed by repeated boosts.

In some embodiments, the nucleic acid vaccinated subject is tested for the presence of IgG1 and/or IgG3 antibodies indicative of an ability to activate complement and clear the virus through complement mediated mechanisms. In one embodiment, the vaccinated subject is tested for the presence of IgG2a, 3 and/or IgM indicative of the ability of the vaccinated subject to produce a functional antibody response rather than non-functional antibodies.

In some embodiments, antibodies generated against in vivo expressed E2 polypeptide include those that at least partially or substantially neutralize an important part of the HCV life cycle such as host cell invasion or viral budding. Functional antibodies may exhibit their life cycle blocking effect by promoting phagocytosis or cytotoxicity or complement mediated clearance.

In one embodiment the description provides for the use a nucleic acid molecule encoding a variable domain deleted E2 polypeptide of HCV in, or in the manufacture of a nucleic acid based medicament for, the treatment or prevention of HCV infection. The term “manufacture” includes production or screening.

In another embodiment, the present disclosure provides a method of eliciting a humoral immune response in a subject or patient, the method comprising administering to the subject an effective amount of a nucleic acid molecule capable of expressing a variable domain deleted E2 polypeptide of HCV.

In one embodiment, the variable deleted E2 is Delta123. In one embodiment, the variable deleted E2, such as E2 Delta 123, has at least four mutated or disrupted cysteines selected from C581 to C620.

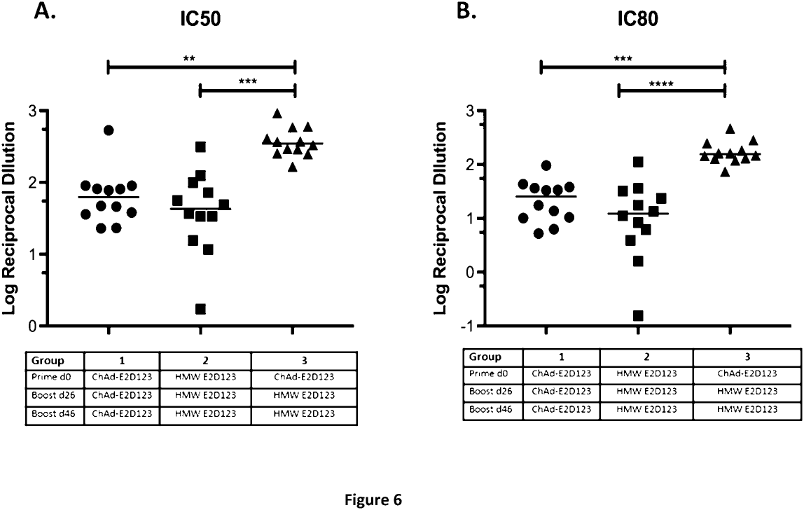

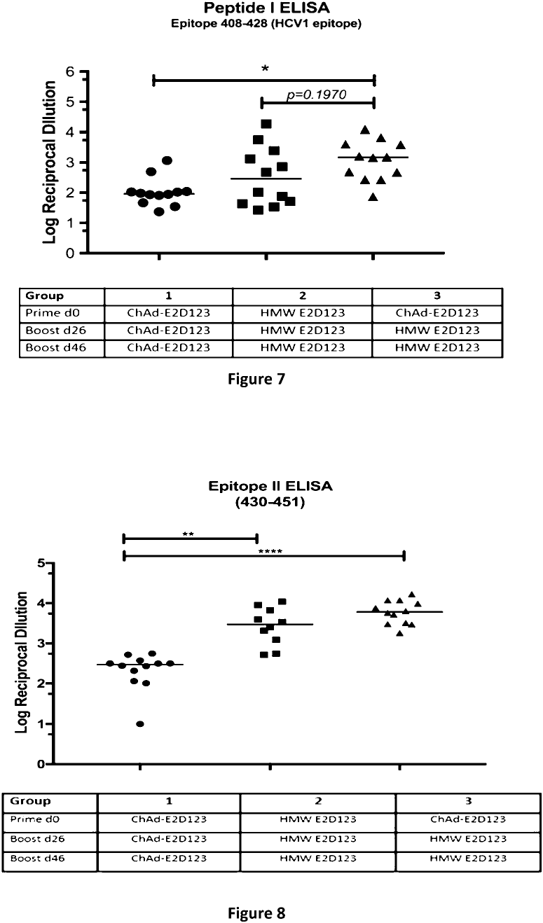

In a related aspect, the present disclosure provides a method of vaccinating a population of subjects against HCV comprising administering to the subject an effective amount of a nucleic acid molecule capable of expressing a variable domain deleted E2 polypeptide of HCV and wherein the vaccine produces a substantially uniform antibody response within the population measurable for example by variability between subject in functional antibody titre as shown in FIG. 6, or by peptide binding as shown in FIGS. 7 or 8 or 9.

In a related embodiment, the present invention provides a method for treating hepatitis C infection in a subject or for immunizing a subject against hepatitis C infection comprising administering to the subject an effective amount of a composition comprising a nucleic acid molecule encoding a variable domain deleted E2 polypeptide of HCV.

The terms “effective amount” including “therapeutically effective amount” and “prophylactically effective amount” as used herein mean a sufficient amount of a composition of the present invention either in a single dose or as part of a series or slow release system which provides the desired therapeutic, preventative, or physiological effect in at least some subjects. Undesirable effects, e.g. side effects, may sometimes manifest along with the desired therapeutic effect; hence, a practitioner balances the potential benefits against the potential risks in determining an appropriate “effective amount”.

The exact amount of composition required will vary from subject to subject, depending on the species, age and general condition of the subject, mode of administration and the like. Thus, it may not be possible to specify an exact ‘effective amount’. However, an appropriate ‘effective amount’ in any individual case may be determined by one of ordinary skill in the art using routine skills or experimentation. One of ordinary skill in the art would be able to determine the required amounts based on such factors as prior administration of the compositions or other agents, the subject’s size, the severity of a subject’s symptoms or the severity of symptoms in an infected population, viral load, and the particular composition or route of administration selected.

The term “treatment” refers to any measurable or statistically significant amelioration in at least some subjects in one or more symptoms of HCV or in the risk of developing advanced symptoms of HCV or the risk of transmitting HCV.

The terms “prevention” and “prophylaxis” and the like are used interchangeably and include administration of a composition of the present invention to a subject not known to be infected with HCV for the purpose of prevention or attenuating a subsequent infection or reducing the risk of becoming infected or reducing the severity or onset of a condition or signs of a condition associated with HCV infection.

The administration of the vaccine composition is generally for prophylactic purposes. The prophylactic administration of the composition serves to prevent or attenuate any subsequent infection. A “pharmacologically acceptable” composition is one tolerated by a recipient patient. It is contemplated that an effective amount of the vaccine is administered. An “effective amount” is an amount sufficient to achieve a desired biological effect such as to induce enough humoral immunity to block at least one stage of the life cycle. This may be dependent upon the type of vaccine, the age, sex, health, and weight of the recipient. Examples of desired biological effects include, but are not limited to, production of no symptoms, reduction in symptoms, reduction in virus titre in tissues or nasal secretions, complete protection against infection by hepatitis C virus, and partial protection against infection by hepatitis C virus, improved herd immunity, protection against the consequences of chronic infection such as hepatitis, hepatocellular cancer etc.

Typically, for viral vectors, about 5 × 10⁷ to 5 × 10¹² viral particles are administered, typically about 5 × 10⁹ to 5× 10¹⁰ viral particles.

Suitable doses for active agent mRNAs encoding Delta123HCV E2 may range, without being limited thereto, from about 10 ng to 1 g, 100 ng to 100 mg, 1 microgram to 10 micrograms or 30-300 micrograms mRNA per patient. In one embodiment, the composition is formulated accordingly to comprise one dose, two doses, three or even more doses.

Suitable dosage ranges for intravenous administration of the nucleic acid molecule encoding HVC E2 are generally about 0.001 to 10 micrograms nucleic acid. Suitable dosage ranges for intranasal administration are generally about 0.01 pg/kg body weight to 10 mg/kg body weight. Effective doses may be extrapolated from dose-response curves derived from in vitro or animal model test systems. Suppositories generally contain active ingredient in the range of 0.5% to 10% by weight; oral compositions preferably contain 10% to 95% active ingredient.

In some aspects, the administration of the nucleic acid vaccine and/or booster administrations may be repeated and such administrations may be separated by at least 1 day, 2 days, 3 days, 4 days, 5 days, 10 days, 15 days, 30 days, 45 days, 2 months, 75 days, e.g. 1 to 5 days, 1 to 10 days, 5 to 15 days, 10 to 20 days, 15 to 25 days, 20 to 30 days, 25 to 35 days, 30 to 50 days, 40 to 60 days, 50 to 70 days, 1 to 75 days, or 1 month, 2 months, 3 months, 4 months, 5 months, or at least 6, 7, 8, 9, 10, 11, 12 months, 18 months, 24 months, 30 months, 36 months, 1 year, 2 years, 3 years, 5 years, 10 years, 15 years, 20 years, 30 years, 40 years, 50 years, 60 years, or even more. In certain aspects, the inventive vaccine may be administered to a subject as a single dose once per year.

In particular aspects, the nucleic acid vaccine may be administered at least once, preferably twice or more to an infected subject prior to administration of direct anti-viral drug treatment, for example at least 1 day, 2 days, 3 days, 4 days, 5 days, 10 days, 15 days, 30 days, 45 days, 2 months, 75 days, e.g. 1 to 5 days, 1 to 10 days, 5 to 15 days, 10 to 20 days, 15 to 25 days, 20 to 30 days, 25 to 35 days, 30 to 50 days, 40 to 60 days, 50 to 70 days, 1 to 75 days, or 1 month, 2 months, 3 months, 4 months, 5 months, or at least 6, 7, 8, 9, 10, 11, or 12 months prior to anti-viral drug therapy. A second or further dose may then be administered directly prior to treatment, concurrent with or subsequent to treatment.

In some embodiments, a vaccine or composition of the present invention is physiologically significant if its presence results in a detectable change in the physiology of a recipient patient that enhances or indicates an enhancement in at least one primary or secondary humoral response against at least one strain of an infectious hepatitis C virus. The vaccine composition is administered to protect against viral infection. The “protection” need not be absolute, i.e., the hepatitis C infection need not be totally prevented or eradicated, if there is a statistically significant improvement compared with a control population or set of patients. Protection may be limited to reducing the severity or rapidity of onset of symptoms of the hepatitis C virus infection.

In one embodiment, a vaccine composition of the present invention is provided to a subject either before the onset of infection (so as to prevent or attenuate an anticipated infection) or after the initiation of an infection, and thereby protects against viral infection. In some embodiments, a vaccine composition of the present invention is provided to a subject before or after onset of infection, to reduce viral transmission between subjects.

It will be further appreciated that compositions of the present invention can be administered as the sole active pharmaceutical agent, or used in combination with one or more agents to treat or prevent hepatitis C infections or symptoms associated with HCV infection. Other agents to be administered in combination with a composition or a combination of compositions of the present invention include therapies for disease caused by HCV infection or that suppress HCV viral replication by direct or indirect mechanisms. These agents include, but are not limited to, host immune modulators (for example, interferon-alpha, pegylated interferon-alpha, consensus interferon, interferon-beta, interferon-gamma, CpG oligonucleotides and the like); antiviral compounds that inhibit host cellular functions such as inosine monophosphate dehydrogenase (for example, ribavirin and the like); cytokines that modulate immune function (for example, interleukin 2, interleukin 6, and interleukin 12); a compound that enhances the development of type 1 helper T cell response; interfering RNA; anti-sense RNA; vaccines comprising HCV antigens or antigen adjuvant combinations directed against HCV; agents that interact with host cellular components to block viral protein synthesis by inhibiting the internal ribosome entry site (IRES) initiated translation step of HCV viral replication or to block viral particle maturation and release with agents targeted toward the viroporin family of membrane proteins such as, for example, HCV P7 and the like; and any agent or combination of agents that inhibit the replication of HCV by targeting other proteins of the viral genome involved in the viral replication and/or interfere with the function of other viral targets, such as inhibitors of NS3 NS4A protease, NS3 helicase, NS5B polymerase, NS4A protein and NS5A protein.

According to yet another embodiment, the pharmaceutical compositions of the present invention may further comprise other inhibitor(s) of targets in the HCV life cycle, including, but not limited to, helicase, polymerase, metal loprotease, NS4A protein, NS5A protein, and internal ribosome entry site (IRES).

Administration is generally for a time and under conditions sufficient to elicit an immune response comprising the generation of functional E2-specific antibodies. The immunogenic compositions may be administered in a convenient manner such as by the pulmonary, oral, intravenous (where water soluble), intraperitoneal, intramuscular, subcutaneous, intradermal, intrathecal or suppository routes or implanting (e.g. using slow release, formulations). Administration may be systemic or local, although systemic is more convenient. Other contemplated routes of administration are by patch, cellular transfer, implant, sublingually, intraocularly, topically, orally, rectally, vaginally, nasally or transdermally. Administration may be in vivo or ex vivo, in vitro. Methods utilizing electroporation, gene guns, ultrasound or high-pressure injection can, for example, be applied for direct delivery of nucleotides to the cells.

As used herein, an “immune response” refers to the reaction of the body as a whole to the presence of a composition of the present invention which includes making antibodies and developing immunity to the composition. Therefore, an immune response to an antigen also includes the development in a subject of a humoral and/or cellular immune response to the antigen of interest. A “humoral immune response” is mediated by antibodies produced by plasma cells. A “cellular immune response” is one mediated by T lymphocytes and/or other white blood cells.

Embodiments of the present invention also provide assays for assessing a functional immune response to the composition. The assays may comprise in vivo assays, such as assays to measure antibody responses and delayed type hypersensitivity responses. In an embodiment, the assay to measure antibody responses primarily may measure B-cell function as well as B-cell/T-cell interactions. For the antibody response assay, antibody titres in the blood may be compared following an antigenic challenge. These levels can be quantitated according to the type of antibody, as for example, IgG, IgGl, IgG2, IgG3, IgG4, IgM, IgA or IgD. Also, the development of immune systems may be assessed by determining levels of antibodies and lymphocytes in the blood without antigenic stimulation.

Also, in an embodiment, phenotypic cell assays can be performed to determine the frequency of certain cell types. Peripheral blood cell counts may be performed to determine the number of lymphocytes or macrophages in the blood. Antibodies can be used to screen peripheral blood lymphocytes to determine the percent of cells expressing a certain antigen as in the non-limiting example of determining CD4 cell counts and CD4/CD8 ratios.

In accordance with these embodiments, the composition is generally administered for a time and under conditions sufficient to elicit an immune response comprising the generation of E2-specific antibodies or functional immune response, complement activation, phagocytosis and cytotoxicity etc. The compositions of the present invention may be administered as a single dose or application. Alternatively, the compositions may involve repeat doses or applications, for example the compositions may be administered 2, 3, 4, 5, 6, 7, 8, 9, 10 or more times. Multiple administrations may be administered at more than two month intervals or more frequently.

A “pharmaceutically acceptable carrier and or a diluent” is a pharmaceutical vehicle comprised of a material that is not otherwise undesirable i.e., it is unlikely to cause a substantial adverse reaction by itself or with the active composition. Carriers may include all solvents, dispersion media, coatings, antibacterial and antifungal agents, agents for adjusting tonicity, increasing or decreasing absorption or clearance rates, buffers for maintaining pH, chelating agents, membrane or barrier crossing agents. A pharmaceutically acceptable salt is a salt that is not otherwise undesirable. The agent or composition comprising the agent may be administered in the form of pharmaceutically acceptable non-toxic salts, such as acid addition salts or metal complexes.

For oral administration, the compositions can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. Tablets may contain a binder such as tragacanth, corn starch or gelatin; a disintegrating agent, such as alginic acid; and a lubricant, such as magnesium stearate. If desired, tablets may be sugar-coated or enteric-coated by standard techniques. The active composition can be encapsulated to make it stable to passage through the gastrointestinal tract. See for example, International Patent Publication No. WO 96/11698.

For parenteral administration, the composition may be dissolved in a carrier and administered as a solution or a suspension. For transmucosal or transdermal (including patch) delivery, appropriate penetrants known in the art are used for delivering the composition. For inhalation, delivery uses any convenient system such as dry powder aerosol, liquid delivery systems, air jet nebulizers, propellant systems. For example, the formulation can be administered in the form of an aerosol or mist. The compositions may also be delivered in a sustained delivery or sustained release format. For example, biodegradable microspheres or capsules or other polymer configurations capable of sustained delivery can be included in the formulation. Formulations can be modified to alter pharmacokinetics and biodistribution. For a general discussion of pharmacokinetics, see, e.g., Remington’s (supra). In some embodiments the formulations may be incorporated in lipid monolayers or bilayers such as liposomes or micelles. Targeting therapies known in the art may be used to deliver the agents more specifically to certain types of cells or tissues.

The actual amount of active agent administered and the rate and time-course of administration will depend on the nature and severity of the disease. Prescription of treatment, e.g. decisions on dosage, timing, etc. is within the responsibility of general practitioners or specialists and typically takes into account the condition of the individual patient, the site of delivery, the method of administration and other factors known to practitioners. Examples of techniques and protocols can be found in Remington’s (supra).

Sustained-release preparations that may be prepared are particularly convenient for inducing immune responses. Examples of sustained-release preparations include semipermeable matrices of solid hydrophobic polymers containing the polypeptide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers, and poly-D-(-)-3-hydroxybutyric acid. While polymers such as ethylene-vinyl acetate and lactic acid-glycolic acid enable release of molecules for over 100 days, certain hydrogels release proteins for shorter time periods. Liposomes may be used which are of the small (about 200-800 Angstroms) unilamellar type in which the lipid content is greater than about 30% cholesterol, the selected proportion being adjusted for the optimal therapy.

In another aspect, the present invention provides a kit comprising a nucleic acid molecule encoding a variable domain deleted E2 polypeptide of HCV. The kits or substrates of the present invention are contemplated for use in diagnostic, prognostic, therapeutic or prophylactic applications as well as for use in designing and/or screening HCV E2 binding molecules or HCV receptor binding molecules.

In one embodiment, the present application provides method of producing the subject nucleic acid molecules within or attached to a viral or non-viral vector, the method comprising attaching or incorporating the subject nucleic acid molecules or their complementary sequences into suitable viral or non-viral vectors. Vectors may be stored or frozen prior to use, or incorporated into kits together with instructions for use.

A “variant” of a polypeptide may contain amino acid of substitutions, preferably conservative substitutions, (for example, 1-50, such as 1-25, in particular 1-10, and especially 1 amino acid residue(s) may be altered) when compared to the reference sequence. Suitably such substitutions do not occur in the region of an epitope, and do not therefore have a significant impact on the immunogenic properties of the antigen. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schinner., Principles of Protein Structure, Springer-Verlag). According to such analyses, groups of amino acids may be defined where amino acids within a group exchange preferentially with each other, and therefore resemble each other most in their impact on the overall protein structure. One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and He, (vii) a slightly-polar group, consisting of Met and Cys, (viii) a small-residue group, consisting of Ser, Thr, Asp, Asn, Gly, Ala, Glu, Gin and Pro, (ix) an aliphatic group consisting of Val, Leu, He, Met and Cys, and (x) a small hydroxyl group consisting of Ser and Thr. Protein variants may also include those wherein additional amino acids are inserted compared to the reference sequence, for example, such insertions may occur at 1-10 locations (such as 1-5 locations, suitably 1 or 2 locations, in particular 1 location) and may, for example, involve the addition of 50 or fewer amino acids at each location (such as 20 or fewer, in particular 10 or fewer, especially 5 or fewer). Suitably such insertions do not occur in the region of an epitope, and do not therefore have a significant impact on the immunogenic properties of the antigen. Variants also include those wherein amino acids have been deleted compared to the reference sequence, for example, such deletions may occur at 1-10 locations (such as 1-5 locations, suitably 1 or 2 locations, in particular 1 location) and may, for example, involve the deletion of 50 or fewer amino acids at each location (such as 20 or fewer, in particular 10 or fewer, especially 5 or fewer). Suitably such deletions do not occur in the region of an epitope, and do not therefore have a significant impact on the immunogenic properties of the antigen. The skilled person will recognise that a particular protein variant may comprise substitutions, deletions and additions (or any combination thereof)-Variants preferably exhibit at least about 70% identity, more preferably at least about 80% identity and most preferably at least about 90% identity (such as at least about 95%, at least about 98% or at least about 99%) to the associated reference sequence. Examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol.

The present description is further illustrated by the following information to be read in conjunction with the Figures and Figure legends.

### Example 1

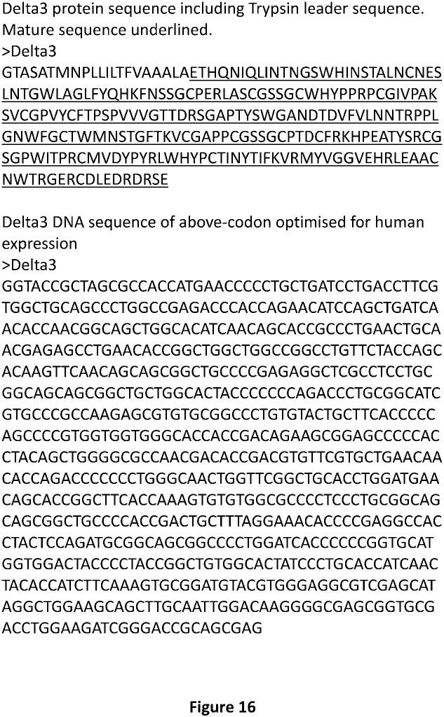

A schematic representation of the immunization and groups of animals used in this study is provided in FIG. 1. Eight week old female C57B16 mice in groups of 12 were immunized with either (Group 1) 10⁸ I.U. Chimpanzee adenovirus 1 encoding the gene for the Genotype 1a E2 D123 sequence (see also FIG. 16) at day 0 (d0) followed by a boost at d26 and d46 with same, or (Group 2) 20ug soluble purified high molecular weight E2 D123 protein at day 0, d26 and d46, or (Group 3) a prime-boost protocol where animals were given a primer of 10⁸ I.U. Chimpanzee adenovirus 1 encoding the gene for the Genotype 1a E2 D123 sequence at day 0 followed by 20ug soluble purified high molecular weight E2 D123 protein boosts at day 26 and day 46. All animals were sacrificed at day 56 and blood and spleens were collected.

The reciprocal antibody titres towards monomeric D123 protein at week (wk) 3 (after prime), week 6 (after prime and one boost) and week 8 (after prime and two boosts) is shown in FIG. 2 The reciprocal dilution was calculated as the dilution of serum required to give 10 times background from a 12 point dilution curve. Data were analysed using a One-way Kruskal-wallis test of multiple comparisons. In this figure, the data is arranged by group to compare the relative antibody titres generated within each protocol over time. The data show that antibody titres peaked at week 8 and were significantly higher than antibody titres at week 3 (group 2 and 3) or week 6 (Group 1).

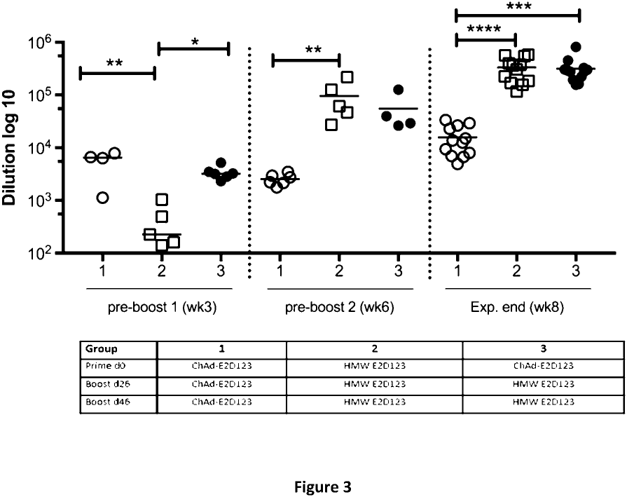

FIG. 3 shows the reciprocal antibody titres towards monomeric D123 protein at week (wk) 3 (after prime), week 6 (after prime and one boost) and week 8 (after prime and two boosts). The reciprocal dilution was calculated as the dilution of serum required to give 10 times background from a 12 point dilution curve. Data were analysed using a One-way Kruskal-Wallis test of multiple comparisons. In this figure, the data is arranged by week to compare the relative antibody titres generated by each vaccination protocol at a given time. The data show that at week 3, animals that received protein alone (group 2) had significantly lower antibody titres compared to group 1 and 3. At week 6 and 8, animals that received ChAd-D123 alone (group 1) had significantly lower antibody titres compared to animals that received protein alone (group 2) or a virus-prime, protein boost (x2) (Group 3). Animals that received three vaccinations with ChAd-E2D123 had the lowest antibody titres at week 6 and 8.

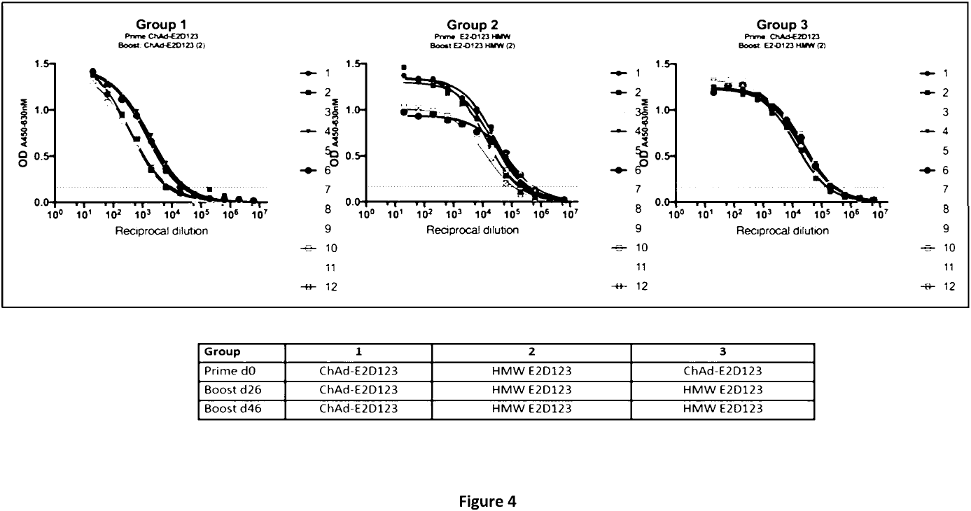

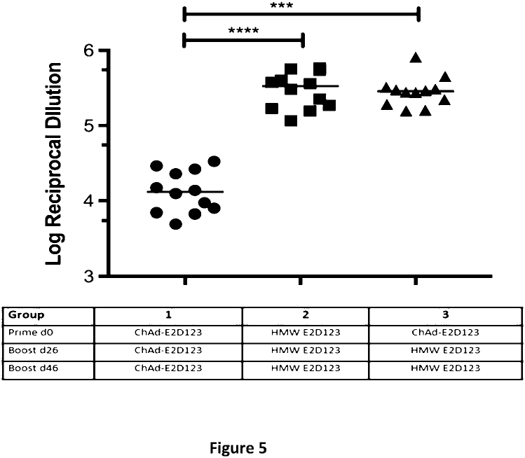

FIG. 4 show the dilution curves of the serum obtained at Day 56 towards monomeric E2 D123 protein. The data show that ChAd-E2D123 priming (Group 1 and 3) generates different titration curves compared to three protein vaccinations (Group 2). However, ChAd-E2D123 prime/boost (group 1) and ChAd-E2D123 prime followed by a protein boosts (group 3) generated more uniform antibody responses in animals.

FIG. 5 shows the reciprocal antibody titres of day 56 serum towards monomeric D123 protein. The data show that the animals that received three vaccinations with ChAd-E2D123 generated significantly lower antibody titres than animals in groups 2 and 3 who received protein only or ChAd-E2D123 prime and two protein boosts, respectively.

FIG. 6 shows the reciprocal dilution of antibody required to inhibit binding of recombinant CD81 protein to intact E2 receptor binding domain by (A) 50% or (B) 80%. The data show that despite group 1 that received three ChAd-E2D123 virus vaccinations eliciting the lowest titres of total antibody reactive to E2 D123, the overall amount of functional antibody able to prevent E2 binding to its cellular receptor CD81 was comparable to group 2 that received three protein vaccinations and that generated almost 100-fold more antibody. The group that generated the highest titres of functional antibody able to prevent E2 binding to its cellular receptor CD81 was group 3 that received one ChAd-E2D123 virus prime followed by two protein boosts. Method for this aspect are described, for example, in Vietheer, P. T.; Boo, I.; Gu, J.; McCaffrey, K.; Edwards, S.; Owczarek, C.; Hardy, M. P.; Fabri, L.; Center, R. J.; Poumbourios, P.; Drummer, H. E., The core domain of hepatitis C virus glycoprotein E2 generates potent cross-neutralizing antibodies in guinea pigs. Hepatology 2017, 65, (4), 1117-1131. In addition, the variation of immune responses was smaller in the ChAd-E2D123 virus prime followed by two protein boosts compared with animals that received three protein vaccinations (group 2) (range 75-470 c.w. 0.15-111, respectively).

FIG. 7 shows the reciprocal titre of antibody directed towards a synthetic peptide comprising amino acids 408-428. This synthetic peptide spans a highly important broadly neutralizing antibody (bNAb) epitope that is recognised by HCV1, MAb24, and other bNAbs. The results show that all vaccination schedules generated antibodies reactive to this peptide with the highest titres observed in animals that either received three protein vaccinations (Group 2) or a ChAd-E2D123 virus prime followed by two protein boosts (group 3) and these were not significantly different to one another.

FIG. 8 shows the reciprocal titre of antibody directed towards a synthetic peptide comprising amino acids 430-451. This synthetic peptide spans a highly important bNAb epitope that is recognised by HCV84.1 and HCV84.27, and other bNAbs. The results show that all vaccination schedules generated antibodies reactive to this peptide with the highest titres observed in animals that either received three protein vaccinations (Group 2) or a ChAd-E2D123 virus prime followed by two protein boosts (group 3) and these were not significantly different to one another.

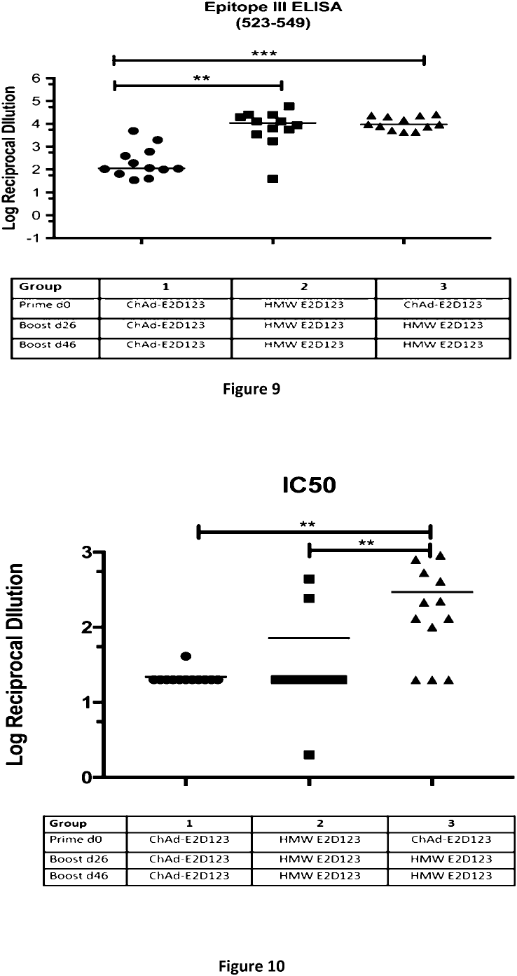

FIG. 9 shows the reciprocal titre of antibody directed towards a synthetic peptide comprising amino acids 523-549. This synthetic peptide spans a highly important neutralizing antibody (NAb) epitope that is recognised by MAb44, and other NAbs and constitutes the CD81 binding loop. The results show that all vaccination schedules generated antibodies reactive to this peptide with the highest titres observed in animals that either received three protein vaccinations (Group 2) or a ChAd-E2D123 virus prime followed by two protein boosts (group 3) and these were not significantly different to one another.

FIG. 10 shows the homologous neutralization titres of day 56 immune serum. The data show that neutralizing antibodies were produced with the most generated in group 3 that received a ChAd-E2D123 virus prime followed by two protein boosts and this was significantly higher than responses observed in groups 1 and 2.

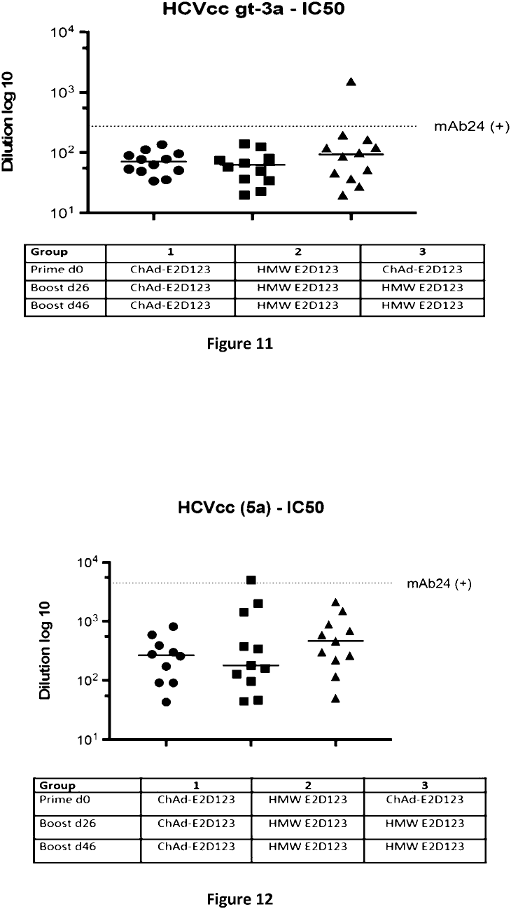

FIG. 11 shows the heterologous neutralization titres of day 56 immune serum against genotype 3a virus. The data show that neutralizing antibodies were produced and were similar in all three groups. This is surprising given that lower antibody titres were generated by group 1 yet they appear to make the same amount of functional antibody as protein only or virus prime-protein boost schedules.

FIG. 12 shows the heterologous neutralization titres of day 56 immune serum against genotype 5a virus. The data show that neutralizing antibodies were produced and were similar in all three groups. This is surprising given that lower antibody titres were generated by group 1 yet they appear to make the same amount of functional antibody as protein only or virus prime-protein boost schedules.

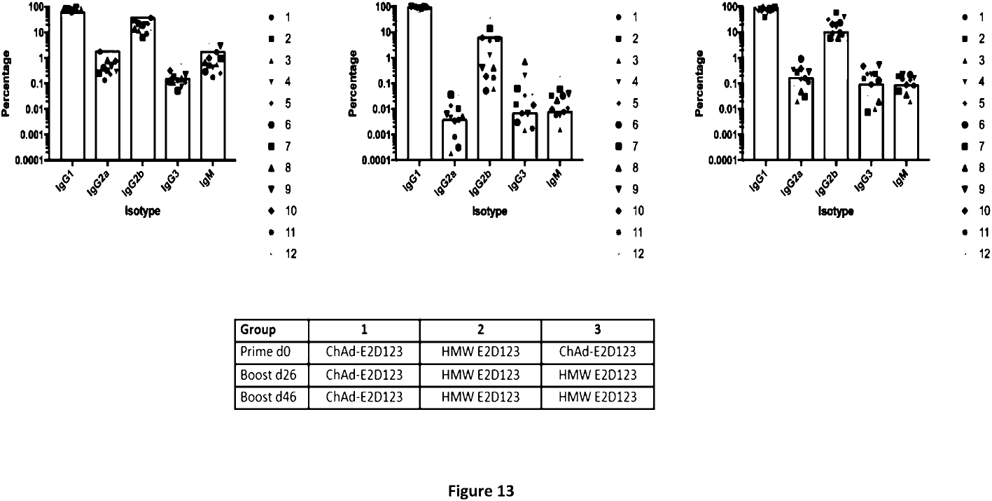

FIG. 13 shows the isotypes of antibodies generated in each of the vaccination schedules. IgG1 was the dominant isotype generated by all vaccines, followed by IgG2b. IgG2a, IgG3 and IgM were minor isotypes. Comparing the percentage of each isotype generated, the data shows that vaccination with ChAd virus either in all three vaccinations (Group 1), or just in the prime (Group 3) generated a higher proportion of IgG2a, 3 and M than protein vaccination alone (Group 2). IgG1 and IgG3 antibodies are more potent activators of complement and this may aid viral clearance. As a percentage IgG3 is really small. It is more than group 2.

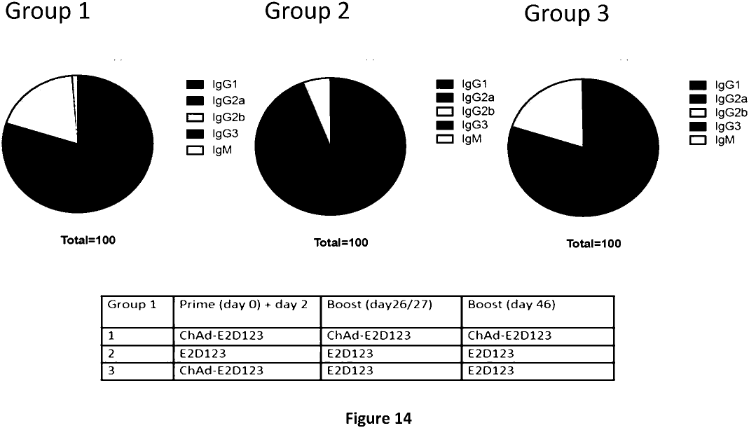

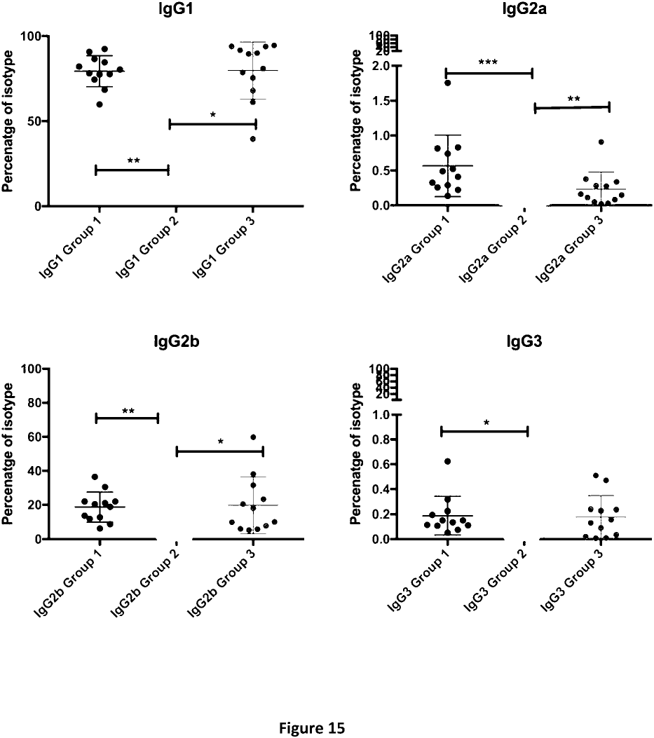

FIG. 14 shows the same data as presented in FIG. 13 as a pie chart for clarity.

FIG. 15 compares the percentage of each isotype generated with each vaccination regimen. The data shows that there was significantly more IgG1 produced in animals vaccinated with soluble HMWD123 in all three vaccinations (Group 2) compared to animals that received D123 as a prime in ChAd vaccine followed by two protein boosts (Group 3) or animals that received all three vaccinations as a ChAd (Group 1). Significantly less IgG2a and IgG2b was produced in animals that received three vaccinations with soluble HMW D123 protein (group 2) than animals that received all three vaccinations as a ChAd (Group 1) or a prime with ChAd-D123 followed by two protein boosts (Group 3). Animals that received three vaccinations with soluble HMW D123 (group 2) had significantly less IgG3 than animals that received three ChAd-D123 vaccinations (group 1).

FIG. 16 shows the protein and DNA sequences of HCV D123 E2 used in this study, for the purpose of illustration only.

### Example 2

Data generated provides further evidence of the generation of cross reactive antibody specificities in animals vaccinated with ChAdD123 then two protein boosts (FIG. 17), as well as higher levels of functional antibody in the animals that received three vaccinations with ChAdD123 (FIG. 19). Also, the data provides evidence of class switching indicative of a maturation of the B cell response. The ChAd-D123 appears to induce a different class switch to protein vaccination alone and this correlates with neutralization (FIG. 18).

FIG. 17 shows the cross-reactive antibody titres at day 56 towards the genotype 3a (S52 isolate) 408-428 epitope I region, 430-451 epitope II region , and 523-549 CD81 binding loop region (epitope III) of HCV E2 generated in animals vaccinated with D123 encoded within Chimpanzee adenovirus (ChAd or ChAdOx1) (Group 1) and boosted with same, or as a high molecular weight soluble protein and boosted with same (Group 2), or a prime with D123 encoded within Chimpanzee adenovirus and boosted with high molecular weight soluble protein (Group 3). * p<0.05, ** p<0.01, *** p<0.001, **** p<0.0001. The data show that a prime with ChAd-E2D123 followed by two protein boosts with HMW E2D123 generates higher titres of cross reactive antibodies to epitope I and III compared with three ChAd-E2D123 vaccinations. Methods of making viral vectors are known in the art and are provided, for example, in von Delft, A.; Donnison, T. A.; Lourenco, J.; Hutchings, C.; Mullarkey, C. E.; Brown, A.; Pybus, O. G.; Klenerman, P.; Chinnakannan, S.; Barnes, E., The generation of a simian adenoviral vectored HCV vaccine encoding genetically conserved gene segments to target multiple HCV genotypes. Vaccine 2018, 36, (2), 313-321.

FIG. 18 shows the IgG2a titre of immune sera calculated as a function of the IgG1 titre and displayed as IgG1 log10 titre divided by IgG2a log10 titre (IgG1/IgG2a). The lower the titre, the closer the IgG2a to IgG1 ratio is to 1:1, indicative of IgG1 to IgG2a class switching. The reciprocal titre (1/IgG1:IgG2a) was plotted against the HCVpp ID50 titre for immune sera from animals that received C/P/P and P/P/P to determine any correlation. All bars are medians and interquartile ranges are displayed. The D′Agostino and Pearson test was used to determine normality of data distribution and Kruskal-Wallis with multiple comparisons were performed to determine significant differences between two group medians at a 95% confidence interval. P values indicate significant difference between groups when <0.05*, <0.01**, <0.001***, <0.0001****. When the IgG2a was expressed as a function of the IgG1 titre (IgG1/IgG2a), ChAd-E2A123 immune sera (groups 1 and 3) had a significantly lower reciprocal titre compared to E2Δ123_{HMW} immune sera indicative of increased GC class switching (p<0.0001**** for group-1 v. group-2, p=0.0014** for group-2 v. group-3). For E2Δ123_{HMW} immune sera (group-2 and -3 combined), the IgG1:IgG2a reciprocal titre positively correlated with HCVpp neutralising titres (r=0.3974, p=0.0492).

FIG. 19 shows the reciprocal ID50 inhibitory titre of immune sera as a function of the overall Ab titre, denoted functional antibody index. The functional antibody index was calculated for both E2-CD81 inhibition shown as CD81 blockade. The functional antibody index was calculated for inhibition of virus entry of homologous pseudotyped virus shown as neutralization. When the reciprocal ID50 titres (CD81 inhibition and HCVpp) are interpreted in relationship to the overall Ab titre (functional antibody index), the ChAd-E2A123 immune sera (group-1 and -3) had a significantly higher functional index compared to E2Δ123_{HMW} immune sera indicating a greater neutralising capacity of the vaccine-induced Ab response relative to the total Ab titre.

Example 3 - HCV D123 E2 can also be successfully delivered for immunisation in a MVA viral vector.

FIG. 20 shows the schematic of the viral vectors used to drive expression of E2 D123. A. Schematic of the expression cassette for D123 in ChAdOx1. B. Schematic of the expression cassette for RBD in ChAdOx1. C. Schematic of the expression cassette for D123 in the MVA. These expression systems are illustrative and variations are known in the art. Inducible expression systems may also be employed as known in the art. Illustrative methods are provided in the art including, von Delft, A.; Donnison, T. A.; Lourenco, J.; Hutchings, C.; Mullarkey, C. E.; Brown, A.; Pybus, O. G.; Klenerman, P.; Chinnakannan, S.; Barnes, E., The generation of a simian adenoviral vectored HCV vaccine encoding genetically conserved gene segments to target multiple HCV genotypes. Vaccine 2018, 36, (2), 313-321; and Swadling, L.; Capone, S.; Antrobus, R. D.; Brown, A.; Richardson, R.; Newell, E. W.; Halliday, J.; Kelly, C.; Bowen, D.; Fergusson, J.; Kurioka, A.; Ammendola, V.; Del Sorbo, M.; Grazioli, F.; Esposito, M. L.; Siani, L.; Traboni, C.; Hill, A.; Colloca, S.; Davis, M.; Nicosia, A.; Cortese, R.; Folgori, A.; Klenerman, P.; Barnes, E., A human vaccine strategy based on chimpanzee adenoviral and MVA vectors that primes, boosts, and sustains functional HCV-specific T cell memory. Sci Transl Med 2014, 6, (261), 261ra153.

FIG. 21 shows the immunization schedule for animals receiving ChAd-E2A123 prime followed by a MVA-E2Δ123 boost at week 4. Animals were bled two weeks after the boost and antibody and T cell reactivity measured. Animals received a week-0 ChAd-E2D123 prime (10⁸ infectious units [IU] in 40uL sterile PBS) followed by a week-4 MVA-E2D123 (10⁷ plaque forming units [PFU] in 40uL sterile PBS ).

FIG. 22A shows the reactivity of immune serum raised to immunization with ChAd-E2Δ123/MVA-E2Δ123 as shown in FIG. 21. For the avoidance of doubt the Delta symbol “Δ” or the word Delta are used interchangeably to refer to “deletion”. Immune serum was assessed in ELISA against E2Δ123 monomer. Serial dilutions of immune serum was applied to E2Δ123 monomer coated plates and bound antibody detected with anti-mouse immunoglobulins conjugated to horse radish peroxidase and TMD substrate. The reciprocal titre required to achieve 10 times background binding was calculated. The results show that a ChAd-E2Δ123/MVA-E2Δ123 generates high titre antibodies reactive to E2. FIG. 22B shows the ability of immune sera generated at day 56 to inhibit the interaction between HCV cellular receptor CD81 and the E2 receptor binding domain. The ability of the immune serum to inhibit 50% of E2 binding CD81 was calculated from serial dilution curves. The results show that a ChAd-E2Δ123/MVA-E2Δ123 generates high titre antibodies capable of inhibiting the binding between homologous E2 and CD81.

FIG. 23A shows the reactivity of immune serum raised to immunization with ChAd-E2Δ123/MVA-E2Δ123 as shown in FIG. 21 to AS412 (E2₄₀₈₋₄₂₈), AS434 (E2₄₃₀₋₄₅₁), CD81 binding loop (E2₅₂₃₋₅₄₉). The reciprocal titre required to achieve 10 times background binding was calculated from serial dilution curves. The results show that all animals generated antibodies capable of binding to three different epitopes that are targets of broadly neutralizing antibodies. FIG. 23B shows the homologous (G1a) and heterologous (G3a) neutralization titres of immune serum raised ChAd-E2Δ123/MVA-E2Δ123 (FIG. 21). The reciprocal titre of immune serum required to inhibit 50% virus entry was was calculated from serial dilution curves. The results show that 7/10 animals generated antibodies capable of preventing entry of homologous genotype 1a hepatitis C virus into a Huh7 cell line and 6/10 animals generated antibodies capable of preventing entry of a heterologous genotype 3a virus into a Huh7 liver cell line. The limit of detection is shown by a dotted line. The cross-neutralizing antibody response towards genotype 3a is consistent with the observation that cross-reactive antibodies towards AS412 (E2₄₀₈₋₄₂₈), AS434 (E2₄₃₀₋₄₅₁), CD81 binding loop (E2₅₂₃₋₅₄₉) were generated by vaccinating animals with ChAd-E2Δ123/MVA-E2Δ123.

FIG. 24 shows the T cell responses in mice immunized with ChAd-E2Δ123/MVA-E2Δ123 as shown in FIG. 21. Splenocytes were harvested at week-6 and stimulated ex vivo using HCV peptides (15mer overlapping by 11aa) covering the length of the HCV proteome (A and C) and for peptide pools (B and D): Core-E1-E2, NS3-4, and NS5, for gt-1a (H77), -1b (J4), or -3a (k3a650). IFNg⁺ CD4⁺ (A and B) and IFNg⁺ CD8⁺ (C and D) T-cell frequencies as a percentage of total CD4⁺ or CD8⁺ T cell frequencies, respectively, were determined via intracellular cytokine staining and flow cytometry. All data are plotted as medians and interquartile ranges. The results show that animals generated both CD4+ and CD8+ T cells towards the homologous peptides, focussed on epitopes within the coreE1E2 region, likely E2. While 2/7 animals tested possessed cross reactive CD4+ T cell responses towards genotype 1b, no CD8+ cross reactivity was observed.

FIG. 25 shows the E2 specific T cell responses in mice immunized with ChAd-E2Δ123/MVA-E2Δ123 as shown in FIG. 21. Splenocytes were harvested at week-6 and stimulated ex vivo using E2 peptide pool from genotype 1a (15mer overlapping by 11aa) covering the length of the E2 region. The data show that mice vaccinated with ChAd-E2Δ123/MVA-E2Δ123 generate E2 specific IFNg+ T cells.

FIG. 26 shows the analysis of polyfunctional T cell responses in mice immunized with ChAd-E2Δ123/MVA-E2Δ123 as shown in FIG. 21. Vaccine-induced T-cell polyfunctionality was determined via ICS and flow cytometry after splenocyte stimulation ex vivo using HCV peptides (15mer overlapping by 11aa) covering the length of the HCV proteome for gt-1a (H77) to detect produced cytokines, IFNg, TNFa, and IL-2. Pie bases are medians and calculated using Pestle and SPICE software. All data are plotted as medians and interquartile ranges. The results show that vaccination with ChAd-E2Δ123/MVA-E2Δ123 generates highly polyfunctional CD4+ and CD8+ T cell responses. CD4 responses were characterized by the production of all 3 cytokines, while CD8+ responses were mostly towards TNFalpha.

All documents cited or referenced herein, and all documents cited or referenced in herein cited documents, together with any manufacturer’s instructions, descriptions, product specifications, and product sheets for any products mentioned herein or in any document incorporated by reference herein, are hereby incorporated herein by reference in their entirety

Those of skill in the art will appreciate that, in light of the instant disclosure, various modifications and changes can be made in the particular embodiments exemplified without departing from the scope of the present invention. All such modifications and changes are intended to be included within the scope of the appended claims. The present embodiments are, therefore, to be considered in all respects as illustrative and not restrictive.

### BIBLIOGRAPHY

Drummer et al., Microbiol. 5:329, 2014.

McCaffrey et al. J. Virol. 81:9584-9590, 2007.

Drummer et al, J. Virol. 76:11143-11147, 2002.

McCaffrey et al. 2012.

Liu et al (2015), Current Drug Metabolism, 16(2):152-165.

Youn et al (2015), Expert Opinion on Biological Therapy, 15(9): 1337-134

Vietheer P.T., et al., Hepatology 65(4), 2017von Delft, A. et al, Vaccine 2018, 36, (2), 313-321

Swadling, L.; Sci Transl Med 2014, 6, (261), 261ra153.

1. A composition comprising a nucleic acid molecule encoding a variable domain deleted E2 polypeptide of HCV for use, or when used, in the treatment or prevention of HCV infection, wherein the use comprises administering the nucleic acid to a subject and wherein the variable domain deleted E2 protein is produced in the subject and generates an immune response including a functional B-cell response to HCV in the subject.

2. The composition for use of claim 1, wherein the nucleic acid molecule encoding a variable domain deleted E2 of HCV is contained within a viral or non-viral vector for vaccination.

3. The composition for use of claim 1, wherein nucleic acid molecule encoding a variable domain deleted E2 of HCV is RNA or DNA or a modified or synthetic form thereof.

4. The composition for use of claim 2 wherein the viral vector is an adenoviral vector, or a poxviral vector.

5. The composition for use of any one of claims 1 to 4, wherein the nucleic acid molecule encodes a variable domain deleted E2 of HCV that has two or all three surface exposed variable domains deleted (Delta23E2, Delta123E2).

6. The composition for use of any one of claims 1 to 5, wherein the nucleic acid molecule encodes HCV E2 polypeptide having the amino acid sequence set out in SEQ ID NO: 1.

7. The composition for use of any one of claims 1 to 6, wherein the nucleic acid molecule comprises the sequence set out in SEQ ID NO:2.

8. The composition for use of any one of claims 1 to 7, wherein the functional B cell response includes a total antibody titre that is lower than the total antibody titre generated after corresponding administration of high molecular weight variable domain deleted E2 polypeptide (e.g., HMWDelta123).

9. The composition for use of any one of claims 1 to 8, wherein the functional B-cell response is comparable to the functional B-cell response generated after corresponding administration of high molecular weight variable domain deleted E2 polypeptide (e.g., HMWDelta123).

10. The composition for use of any one of claims 1 to 8, wherein the functional immune response comprises CD81 inhibition titres comparable to or higher than the CD81 inhibition titres generated after corresponding administration of high molecular weight variable domain deleted E2 polypeptide (HMWDelta123).

11. The composition for use of any one of claims 1 to 10 further comprising another therapeutically or prophylactically active ingredient.

12. Use of a nucleic acid molecule encoding a variable domain deleted E2 polypeptide of HCV in, or in the manufacture of a nucleic acid or nucleic acid vectored medicament for the treatment or prevention of HCV infection.

13. The use of claim 12, wherein the nucleic acid molecule encoding a variable domain deleted E2 polypeptide of HCV is contained within a viral or non-viral vector. for vaccination.

14. The use of claim 12 or 13, wherein the nucleic acid molecule encoding a variable domain deleted E2 of HCV is RNA or DNA or a modified form thereof.

15. The use of claim 13, wherein the viral vector is an adenoviral vector or a poxviral vector, such as MVA.

16. The use of any one of claims 12 to 15, wherein the nucleic acid molecule encodes a variable domain deleted E2 of HCV that has two or all three surface exposed variable domains deleted (e.g., Delta123E2).

17. The use of any one of claims 12 to 16, wherein the nucleic acid molecule encodes HCV E2 polypeptide having the amino acid sequence set out in SEQ ID NO: 1.

18. The use of any one of claims 12 to 17, wherein the nucleic acid molecule wherein the nucleic acid molecule comprises the sequence set out in SEQ ID NO:2.

19. A method of treatment or prevention of HCV infection, comprising the administration of a composition according to any one of claims 1 to 10 to a subject for a time and under conditions to generate a functional B-cell response to HCV in the subject.

20. The method of claim 19, wherein the composition is administered as a prime vaccination, or as a prime and a boost vaccination.

21. The method of claim 19, wherein the composition is administered as a prime vaccination, followed by two booster vaccinations.

22. The method of claim 19, wherein the composition is administered as a prime vaccination and wherein a booster vaccination comprises high molecular weight variable domain deleted E2 polypeptide of HCV.
